(19) United States
(12) Patent Application Publication
(10) Pub. No.: US 2024/0398234 A1
(43) Pub. Date: Dec. 5, 2024

(54) WEARABLE PULSE OXIMETER AND RESPIRATION MONITOR

(71) Applicant: Masimo Corporation, Irvine, CA (US)

(72) Inventors: Ammar Al-Ali, San Juan Capistrano, CA (US); Chad A. DeJong, Los Angeles, CA (US)

(21) Appl. No.: 18/784,001

(22) Filed: Jul. 25, 2024

Related U.S. Application Data
(63) Continuation of application No. 17/525,140, filed on Nov. 12, 2021, now Pat. No. 12,070,293.
Application No. 17/525,140 is a continuation of application No. 16/813,551, filed on Mar. 9, 2020, now Pat. No. 11,202,571.
Application No. 16/813,551 is a continuation of application No. 15/644,152, filed on Jul. 7, 2017, now Pat. No. 10,617,302.
(60) Provisional application No. 62/359,589, filed on Jul. 7, 2016.
(60) Provisional application No. 62/463,331, filed on Feb. 24, 2017.

(51) Int. Cl. A61B 5/00; A61B 5/1455

(52) U.S. Cl. CPC A61B 5/0059; A61B 5/0015; A61B 5/14552; A61B 5/6824; A61B 5/6828; A61B 5/6829; A61B 5/6831; A61B 5/742; A61B 2560/0443

(57) ABSTRACT

A system for monitoring physiological information of a subject can include a wearable assembly and a monitor instrument. The wearable assembly can include a strap for securing the wearable assembly to the subject, a base connected to the strap, and a pulse oximetry sensor for generating one or more signals indicative of a physiological parameter of the subject. The monitor instrument can removably secure and electrically couple to the base of the wearable assembly and include a processor for determining the physiological parameter of the subject.

### CROSS-REFERENCE TO RELATED APPLICATIONS

This application is a continuation of U.S. patent application Ser. No. 17/525,140, filed Nov. 12, 2021, titled WEARABLE PULSE OXIMETER AND RESPIRATION MONITOR, which is a continuation of U.S. patent application Ser. No. 16/813,551, filed Mar. 9, 2020, titled WEARABLE PULSE OXIMETER AND RESPIRATION MONITOR, now issued as U.S. Pat. No. 11,202,571, which is a continuation of U.S. patent application Ser. No. 15/644,152, filed Jul. 7, 2017, titled WEARABLE PULSE OXIMETER AND RESPIRATION MONITOR, now issued as U.S. Pat. No. 10,617,302, which claims the benefit of U.S. Provisional Application No. 62/359,589, filed Jul. 7, 2016, titled WEARABLE PULSE OXIMETER AND RESPIRATION MONITOR, and U.S. Provisional Application No. 62/463,331, filed Feb. 24, 2017, titled WEARABLE PULSE OXIMETER AND RESPIRATION MONITOR. Each of the foregoing applications is hereby incorporated by reference herein in its entirety.

### FIELD OF DISCLOSURE

In general, the present disclosure relates to a wearable patient monitoring device, and methods and apparatuses for monitoring a patient's physiological information using the device. More specifically, the present disclosure relates to the connection of physiological sensors to instruments responsive to signals from the sensors.

### BACKGROUND

Hospitals, nursing homes, and other patient care facilities typically include patient monitoring devices at one or more bedsides in the facility. Patient monitoring devices generally include sensors, processing equipment, and displays for obtaining and analyzing a medical patient's physiological parameters such as blood oxygen saturation level, respiratory rate, pulse, and a myriad of other parameters, such as those monitored on commercially available patient monitors from Masimo Corporation of Irvine, California. Clinicians, including doctors, nurses, and other medical personnel, use the physiological parameters and trends of those parameters obtained from patient monitors to diagnose illnesses and to prescribe treatments. Clinicians also use the physiological parameters to monitor patients during various clinical situations to determine whether to increase the level of medical care given to patients.

In an embodiment, the patient monitoring devices include a pulse oximeter. Pulse oximetry is a widely accepted noninvasive procedure for measuring the oxygen saturation level of arterial blood, an indicator of a person's oxygen supply. A typical pulse oximetry system utilizes an optical sensor clipped onto a fingertip to measure a relative volume of oxygenated hemoglobin in pulsatile arterial blood flowing within, for example, the fingertip, foot, ear, forehead, or other measurement sites. The oximeter can, in various embodiments, calculate oxygen saturation (SpO₂), pulse rate, a plethysmograph waveform, perfusion index (PI), pleth variability index (PVI), methemoglobin (MetHb), carboxyhemoglobin (CoHb), total hemoglobin (tHb), glucose, and/or otherwise, and the oximeter can display on one or more monitors the foregoing parameters individually, in groups, in trends, as combinations, or as an overall wellness or other index. An example of such an oximeter, which can utilize an optical sensor described herein, are described in U.S. application Ser. No. 13/762,270, filed Feb. 7, 2013, titled “Wireless Patient Monitoring Device,” U.S. application Ser. No. 14/834,169, filed Aug. 24, 2015, titled “Wireless Patient Monitoring Device,” and U.S. application Ser. No. 14/511,974, filed Oct. 10, 2014, titled “Patient Position Detection System,” the disclosures of which are hereby incorporated by reference in their entirety.

The patient monitoring devices can also communicate with an acoustic sensor comprising an acoustic transducer, such as a piezoelectric element. The acoustic sensor can detect respiratory and other biological sounds of a patient and provide signals reflecting these sounds to a patient monitor. An example of such an acoustic sensor, which can implement any of the acoustic sensing functions described herein, is described in U.S. application Ser. No. 12/643,939, filed Dec. 21, 2009, titled “Acoustic Sensor Assembly,” and in U.S. Application No. 61/313,645, filed Mar. 12, 2010, titled “Acoustic Respiratory Monitoring Sensor Having Multiple Sensing Elements,” the disclosures of which are hereby incorporated by reference in their entirety. An example of such an acoustic sensor is also described in U.S. application Ser. Nos. 13/762,270, 14/834,169, and 14/511,974 referenced above.

### SUMMARY OF THE DISCLOSURE

In the present disclosure, one or more sensors can be connected to a wireless monitor configured to receive the sensor data, process the data to determine any number of a myriad of physiological parameters, and wirelessly transmit the sensor data or the physiological parameters responsive to the sensor data to a bedside monitor. The bedside monitor can be configured to output the physiological parameters, communication channel, and/or communication status. An example of methods and apparatuses for wirelessly monitoring a patient's physiological information is described in U.S. application Ser. Nos. 13/762,270, 14/834,169, and 14/511,974 referenced above.

Durable and disposable sensors are often used for the patient monitoring devices. These sensors can have connectors which allow detachment from a monitor instrument or a cable. One example of the connectors can include the use of pogo pins on a pin end and a plurality of electrical contacts on a surface of a sensor end. The pin end can have a plurality of retractable electrical connectors or pogo pins extending through pin holes on a printed circuit board. The plurality of electrical contacts on the sensor end are configured to engage contact tips of the plurality of pogo pins when the pin end comes into close proximity with the sensor end. An example of the pogo pin connectors is described in U.S. application Ser. No. 15/017,349, filed Feb. 5, 2016, titled “Pogo Pin Connector,” which is expressly bodily incorporated in its entirety and is part of this disclosure.

One aspect of the disclosure is a wireless patient monitoring device for measuring one or more parameters that can be secured to a wrist of the patient. The wireless patient monitoring device can include a monitor instrument, a base, and a strap. The monitor instrument can removably mechanically and electrically engage the base. In some embodiments, the monitor instrument can have a display screen. The base can have a strap connector for engaging a strap that can be worn on the patient's wrist. The base can have an outlet on a first end configured to be connected to a first sensor. In some embodiments, the base can also have an outlet on a second end configured to be connected to a second sensor. The first end can be opposite the second end along a length of the base. The base can have a plurality of electrical contacts on an anterior surface. The plurality of electrical contacts can be configured to contact a plurality of pogo pins extending from a posterior surface of the monitor instrument. The contact between the electrical contacts and the pogo pins can electrically connect the monitor instrument to the sensors that are coupled to the base. The monitor instrument can then receive data from one or both sensors, it can process the data to determine responsive parameters/measurements and/or can transmit the data and calculated parameter information wirelessly to a bedside monitor. In some embodiments, one of the sensors is configured to be connected to the base and can comprise a noninvasive optical sensor of the type used in pulse oximetry. In some embodiments, one of the sensors is configured to be connected to the base and can comprise a non-invasive acoustic sensor of the type used in breath sounds monitoring to determine respiration rate and/or cardiac parameters.

A patient monitoring device configured to be removably secured to a patient and responsive to one or more physiological parameters of the patient can comprise a reusable monitor instrument configured to transmit wireless information to a remote patient monitor and having a plurality of electrical connectors extending from a surface of the monitor instrument; and a disposable portion including (a) at least one non-invasive physiological sensor comprising one of an optical sensor and an acoustic sensor, (b) a base having (i) an electrical connector configured to connect to the at least one physiological sensor, the at least one physiological sensor including its own sensor attachment mechanism separate from the disposable portion, said sensor attachment mechanism configured to removably secure said at least one physiological sensor to a measurement site on said patient, and (ii) a plurality of electrical contacts on a surface, the electrical connector including electronics operably connecting the at least one physiological sensor to the plurality of electrical contacts, the monitor instrument configured to removably mechanically engage the base, the electrical connectors configured to electrically contact the electrical contacts, and (c) an attachment mechanism configured for removably securing the base to the patient, wherein the monitor instrument can be responsive to signals from the at least one physiological sensor, said signals responsive to physiological parameters of the patient. The base can further comprise a second electrical connector configured to connect to a second non-invasive physiological sensor. The physiological sensor can comprise the optical sensor. The physiological sensor can comprise the acoustic sensor. The monitor instrument can comprise a display screen. The plurality of electrical connectors can comprise pogo pins. The device can further comprise one or more cable management mechanisms on the reusable monitor instrument or the base, the one or more cable management mechanisms configured to secure sensor cables.

A patient monitoring device configured to be removably secured to a patient and responsive to one or more physiological parameters of the patient can comprise a reusable monitor instrument configured to transmit wireless information to a remote patient monitor and having a plurality of electrical connectors extending from a surface of the monitor instrument; and a disposable portion including (a) at least two non-invasive physiological sensors, each sensor including a sensor positioner configured to position the sensor with respect to a measurement site on said patient, (b) a base having (i) at least first and second electrical connectors configured to connect to the at least two physiological sensors respectively, and (ii) a plurality of electrical contacts on a surface, the electrical connectors including electronics operably connecting the at least two physiological sensors to the plurality of electrical contacts, the monitor instrument configured to removably mechanically engage the base, the electrical connectors configured to electrically contact the electrical contacts, and (c) an attachment mechanism configured for removably securing the base to the patient, wherein the monitor instrument can be responsive to signals from the at least two physiological sensors, said signals responsive to physiological parameters of the patient. The attachment member can comprise a band configured to be removably secured onto the patient's arm, wrist, leg, or ankle. The attachment member can comprise a cord configured to be worn around the patient's neck. The at least first and second electrical connectors can be positioned on the same side of the base. At least first and second electrical connectors can be configured to removably connect the at least two physiological sensors such that the at least first and second electrical connectors can be exchanged. The plurality of electrical connectors can comprise pogo pins. The device can further comprise one or more cable management mechanisms on the reusable monitor instrument or the base, the one or more cable management mechanisms configured to secure sensor cables.

A patient monitoring device configured to be removably secured to a patient and responsive to one or more physiological parameters of the patient can comprise a reusable monitor instrument configured to transmit wireless information to a remote patient monitor and having at least one electrical connector extending from a surface of the monitor instrument, the at least one electrical connector including electronics configured for operably connecting to at least one physiological sensor; and a disposable portion including a base and an attachment mechanism configured for removably securing the base to the patient, the monitor instrument configured to removably mechanically engage the base, wherein the monitor instrument can be responsive to signals from the at least one physiological sensor, said signals responsive to physiological parameters of the patient. The attachment member can comprise a band configured to be removably secured onto the patient's arm, wrist, leg, or ankle. The attachment member can comprise a cord configured to be worn around the patient's neck. The device can further comprise one or more cable management mechanisms on the reusable monitor instrument or the base, the one or more cable management mechanisms configured to secure sensor cables.

### BRIEF DESCRIPTION OF THE DRAWINGS

Various embodiments will be described hereinafter with reference to the accompanying drawings. These embodiments are illustrated and described by example only, and are not intended to limit the scope of the disclosure. In the drawings, similar elements have similar reference numerals.

FIGS. 1A-C illustrate perspective and front views of an embodiment of a wireless patient monitoring device connected to two physiological sensors.

FIGS. 1D-1F illustrate various perspective views of an embodiment of a wireless patient monitoring device connected to two physiological sensors.

FIGS. 1G-1I various perspective views of illustrate an embodiment of a wireless patient monitoring device connected to two physiological sensors.

FIGS. 2A-C illustrate partially exploded views of the embodiment of the wireless patient monitoring device of FIGS. 1A-B connected to two physiological sensors.

FIGS. 2D-E illustrates front and back views of embodiments of pads or printed circuit boards (“PCBs”) having a plurality of electrical contacts for use in an embodiment of the wireless patient monitoring device.

FIG. 2F illustrates back views of a base and a strap of an embodiment of the wireless patient monitoring device.

FIGS. 3A-C illustrate left, front and bottom views of an embodiment of the wireless patient monitoring device.

FIGS. 4A-C illustrate left, front and bottom views of the embodiment of the wireless patient monitoring device of FIGS. 3A-C with internal structures shown in broken lines.

FIGS. 5A-D illustrate perspective, left, front, and bottom views of another embodiment of the wireless patient monitoring device.

FIG. 5E illustrates the embodiment of the wireless patient monitoring device of FIGS. 5A-D connecting to a physiological sensor.

FIG. 5F illustrates another embodiment of the wireless patient monitoring device connecting to a physiological sensor.

FIG. 6A illustrates a partial exploded view of the embodiment of the wireless patient monitoring device of FIGS. 5A-D.

FIGS. 6B-E illustrate steps for disassembling a monitor instrument from a base of the embodiment of the wireless patient monitoring device of FIGS. 5A-D.

FIG. 6F illustrates front views of a base, a strap and a sensor cable of another embodiment of the wireless patient monitoring device.

FIGS. 7A-E illustrate embodiments of the wireless patient monitoring device suitable for wearing on both the patient's left and right wrists.

FIGS. 8A-B illustrate another embodiment of the wireless patient monitoring device that can be worn around a patient's neck.

FIGS. 9A-B illustrate another embodiment of the wireless patient monitoring device that can be worn on the patient's wrist.

FIGS. 10A-B illustrate the embodiments of the wireless patient monitoring device of FIGS. 8A-B and 9A-B attached to a physiological sensor, with the monitor instrument detached from the bases.

FIGS. 11A-D illustrate another embodiment of the wireless patient monitoring device.

FIG. 12 illustrates a patient wearing an example wireless patient monitoring device.

FIG. 13 illustrates a patient wearing an example wireless patient monitoring device.

### DETAILED DESCRIPTION

Although certain embodiments and examples are described below, those of skill in the art will appreciate that the disclosure extends beyond the specifically disclosed embodiments and/or uses and obvious modifications and equivalents thereof. Thus, it is intended that the scope of the disclosure herein disclosed should not be limited by any particular embodiments described below.

In clinical settings, medical sensors are often attached to patients to monitor physiological parameters of the patients. Some examples of medical sensors include, but are not limited to, blood oxygen sensors, such as pulse oximetry sensors, acoustic respiratory sensors, EEGs, ECGs, blood pressure sensors, sedation state sensors, etc. Typically, each sensor attached to a patient is connected to a bedside monitoring device with a cable. The cables limit the patient's freedom of movement and impede a care provider's access to the patient. The cables connecting the patient to the bedside monitoring device also make it more difficult to move the patient from room to room or switch to different bedside monitors.

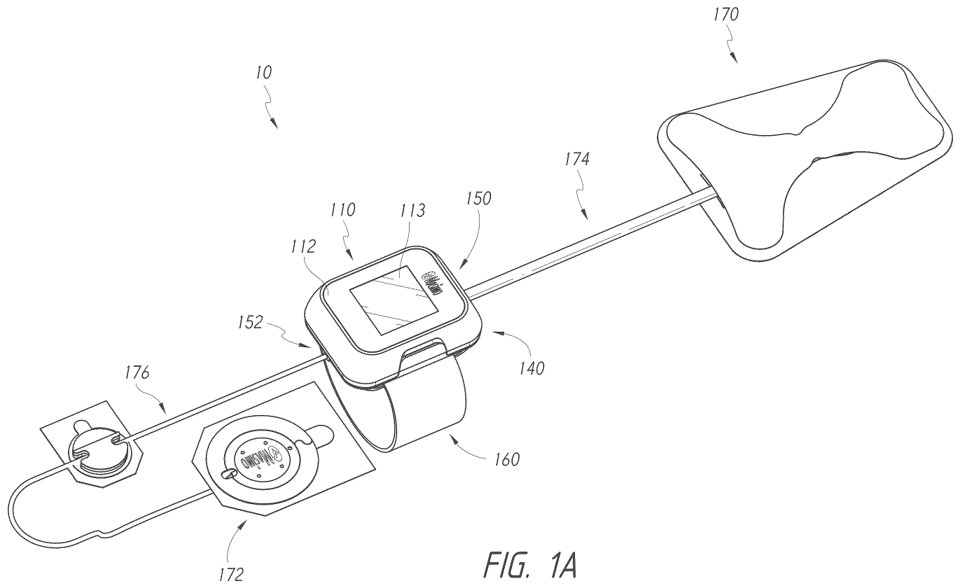

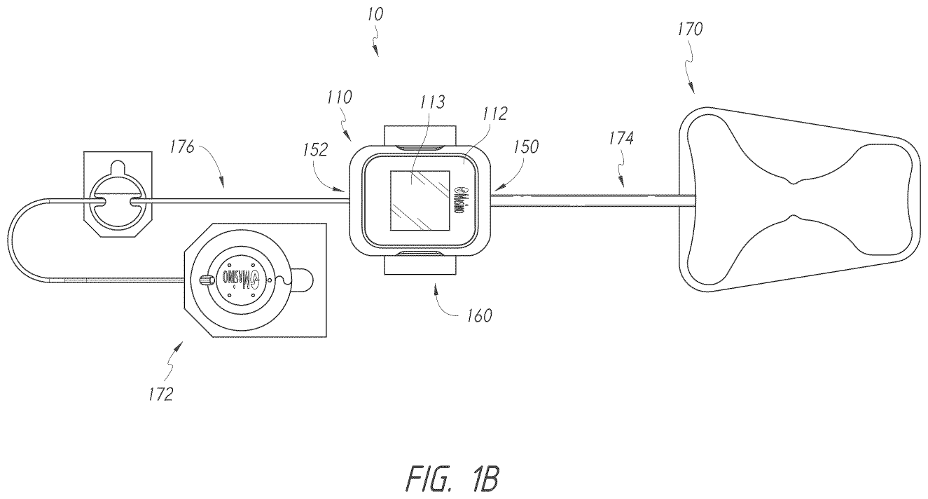

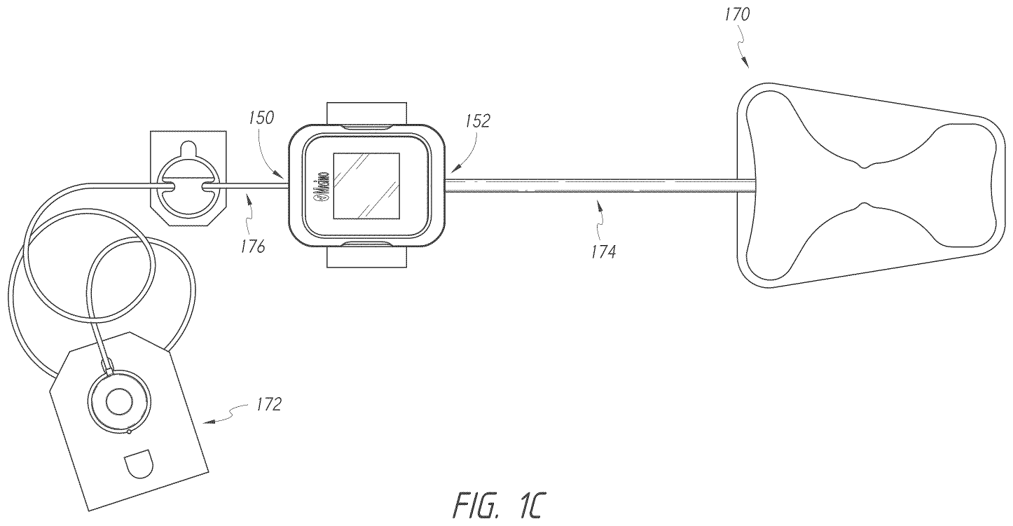

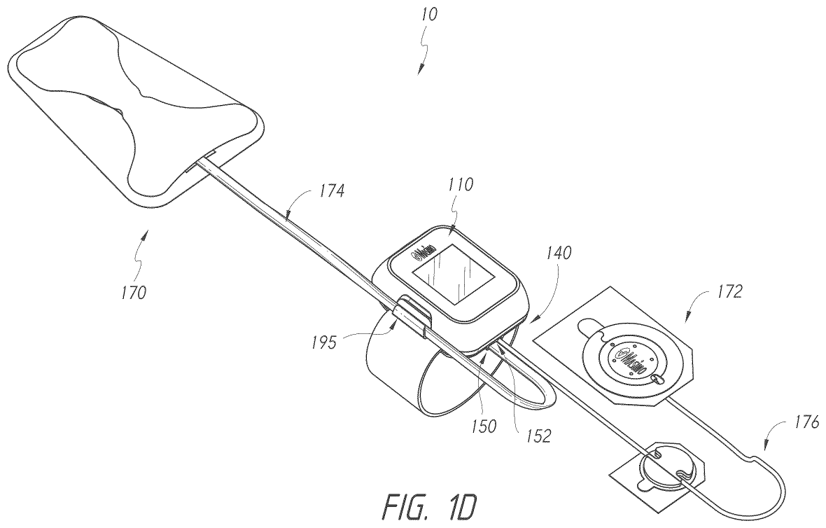

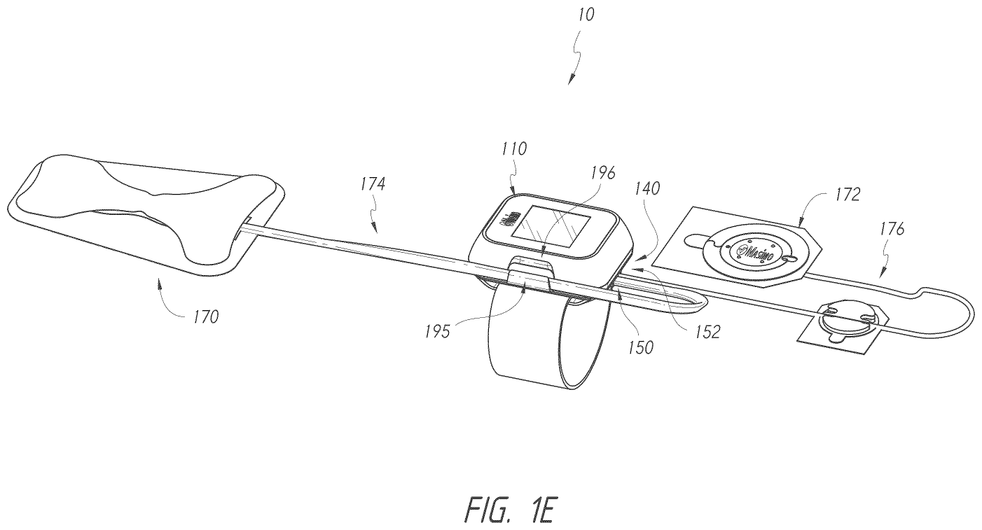

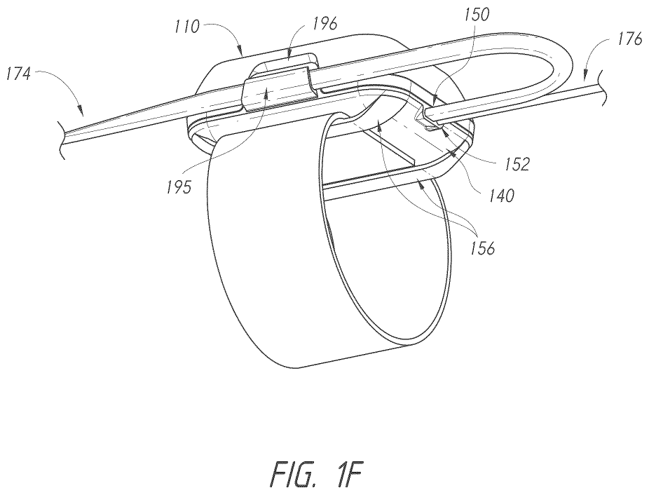

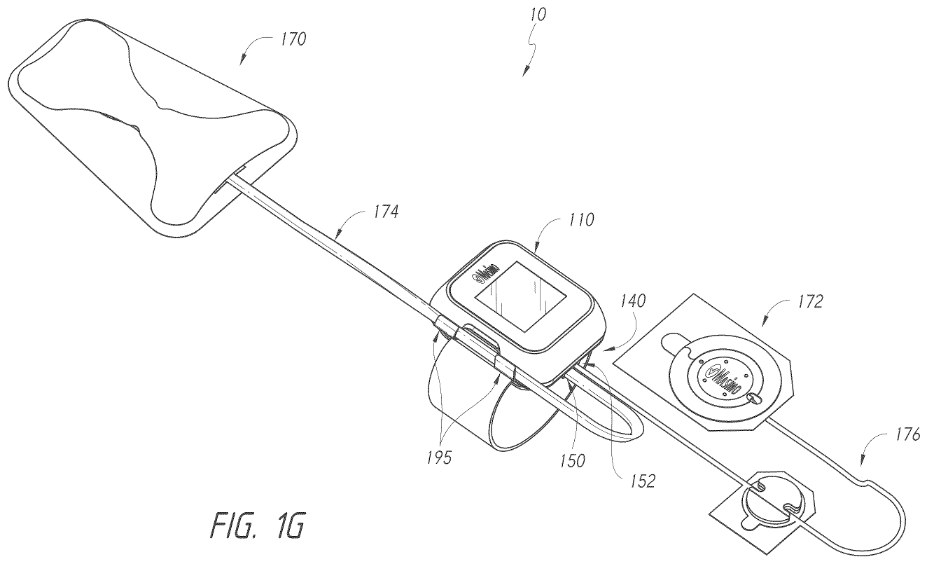

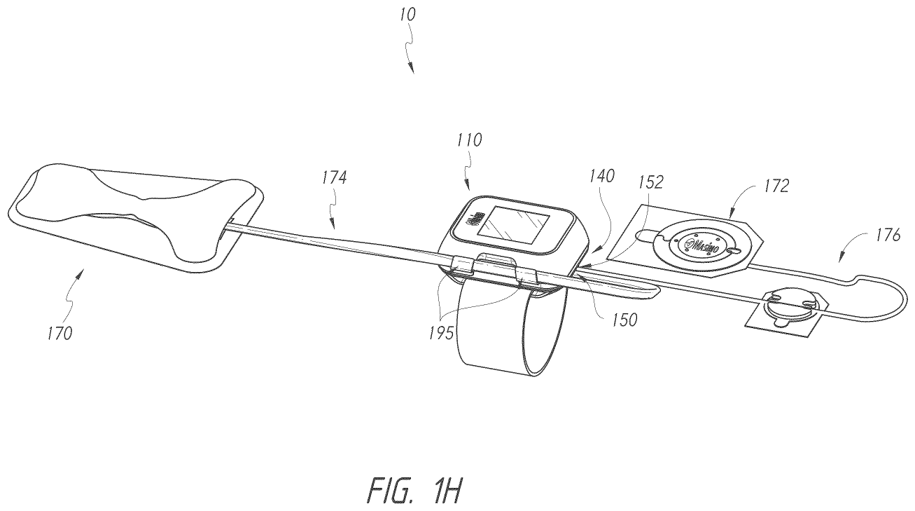

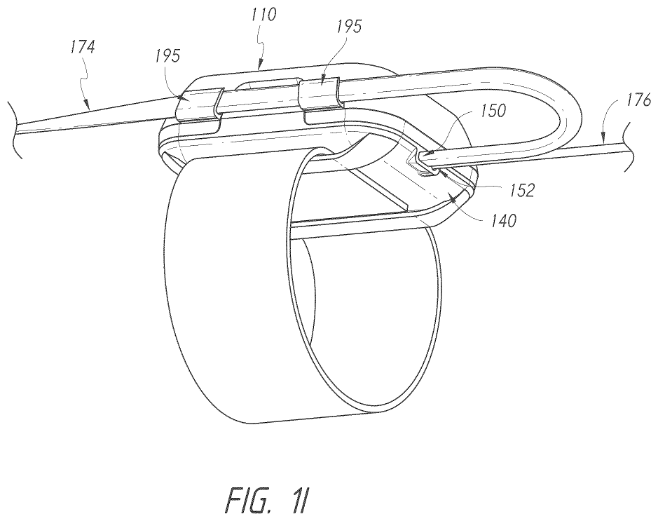

This disclosure describes embodiments of wireless patient monitoring devices that are coupled to one or more sensors and worn by a patient. FIGS. 1A-B illustrate an embodiment of the wireless patient monitoring device 10. The wireless patient monitoring device 10 can have a monitor instrument 110, a base 140, and a strap 160. The monitor instrument 110 can be reusable. The base 140 and/or the strap 160 can be disposable.

The monitor instrument 110 can include a wireless transceiver capable of transmitting data using any of a variety of wireless technologies, such as Wi-Fi (802.11x), Bluetooth (802.15.2), Zigbee (802.15.4), cellular telephony, infrared, RFID, satellite transmission, proprietary protocols, combinations of the same, and the like. The monitor instrument 110 can also include processing capabilities. The monitor instrument 110 can include a hardware processor. The monitor instrument can include a printed circuit board (PCB). In some embodiments, the monitor instrument 100 can have a battery. In some embodiments, the battery can be built inside the monitor instrument 110 and rechargeable. For example, the battery can be recharged when the monitor instrument 100 is placed on a charging dock. In other embodiments, the battery can be replaceable. The monitor instrument 100 can transmit sensor data obtained from sensors to a remote patient monitor (not shown). For example, the remote patient monitor can be a bedside monitor. By transmitting the sensor data wirelessly, the patient monitoring device 10 can advantageously replace some or all cables that connect patients to the bedside monitor. Detailed methods and apparatuses of wirelessly transmitting sensor data to bedside monitoring devices are described in U.S. application Ser. Nos. 13/762,270, 14/834,169, and 14/511,974 referenced above.

An artisan will recognize from the disclosure herein that the device 10 can include additional and/or alternative features and functions. For example, the device 10 can advantageously upload its data to a cloud-based computing platform or data storage platform where the device manufacturer can manage the data, a caregiver, caregiver facility or insurance provider can access the data, or the like. Also, while shown as a device for attachment to the wrist or appendages of non-infants, the device can attach to an ankle of an infant or neonate where the optical sensor is attached to the foot. Other embodiments can use an ear or nose optical sensor, or can combine a nose optical sensor and an acoustic sensor. Still additional embodiments can secure to the head or other site on the body, can include position sensors, fall detection algorithms, patient turn protocols and algorithms or the like.

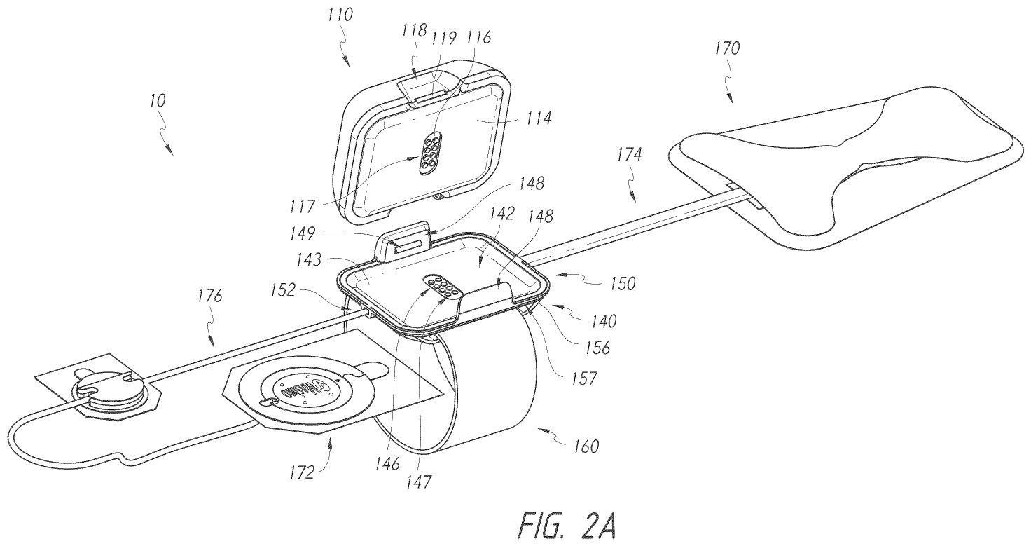

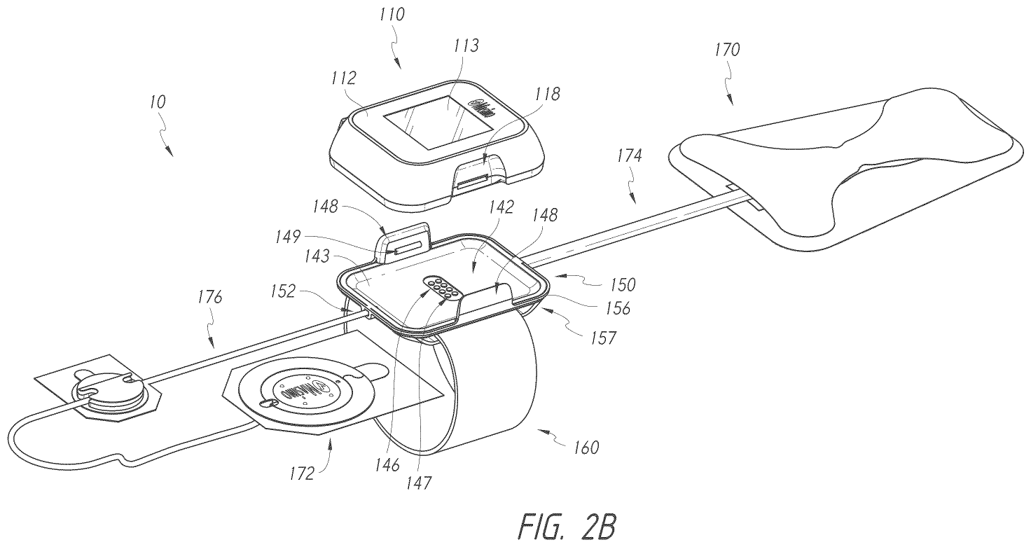

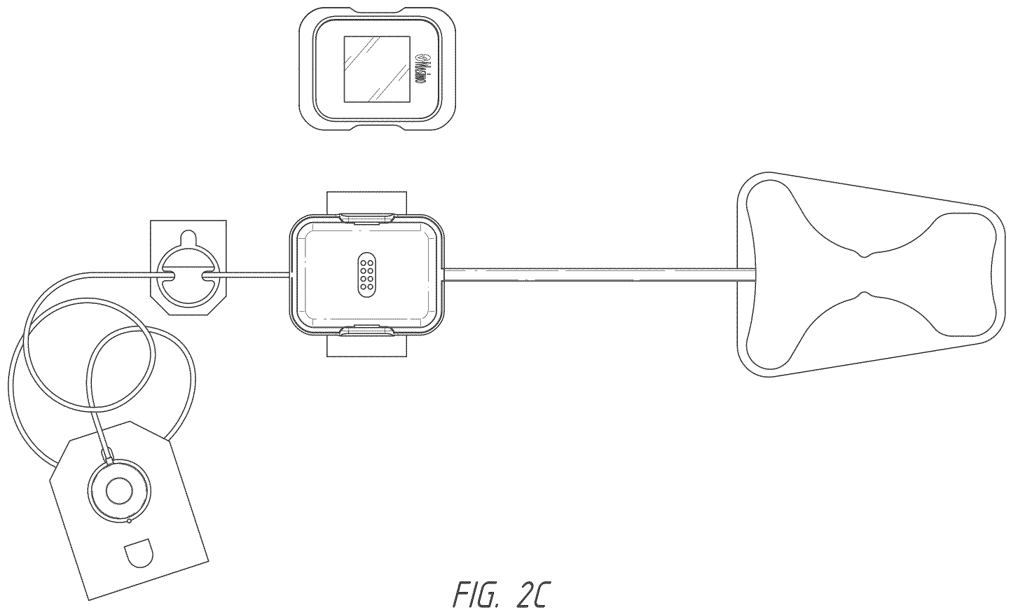

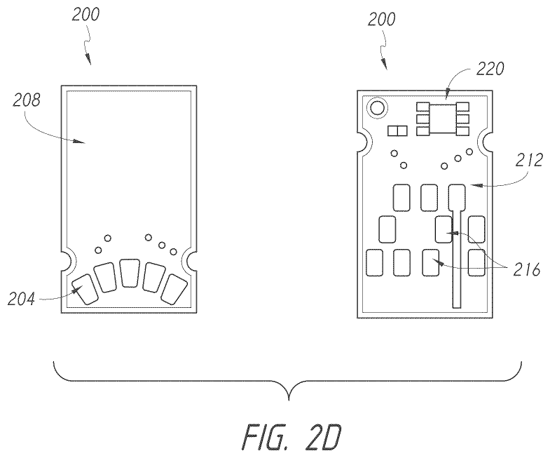

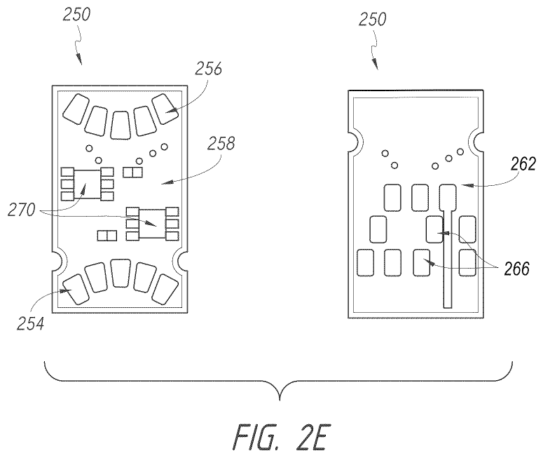

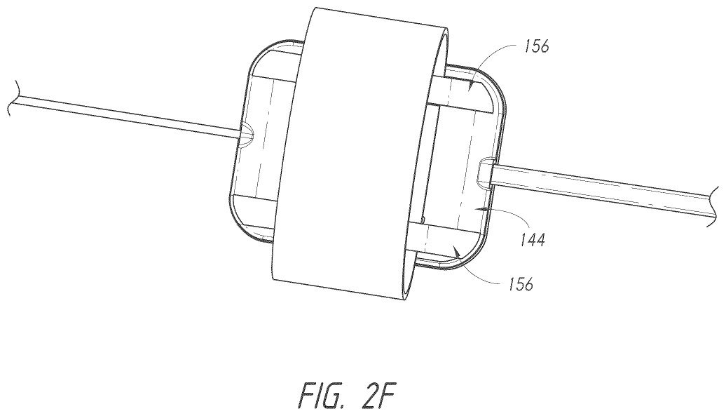

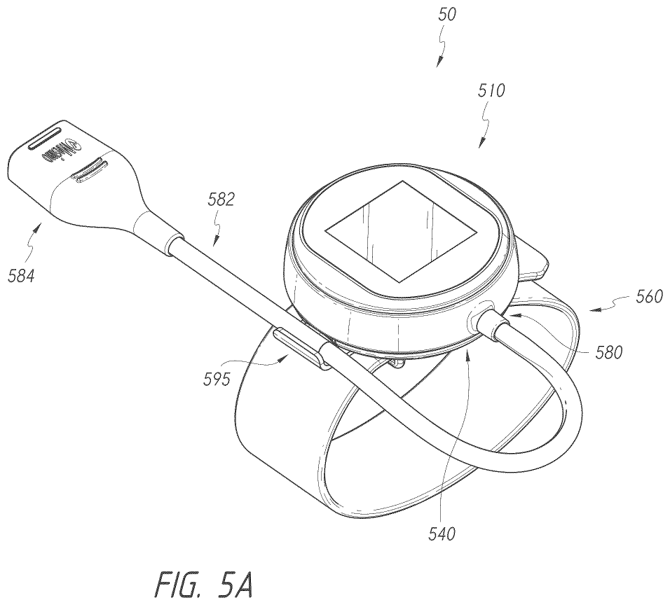

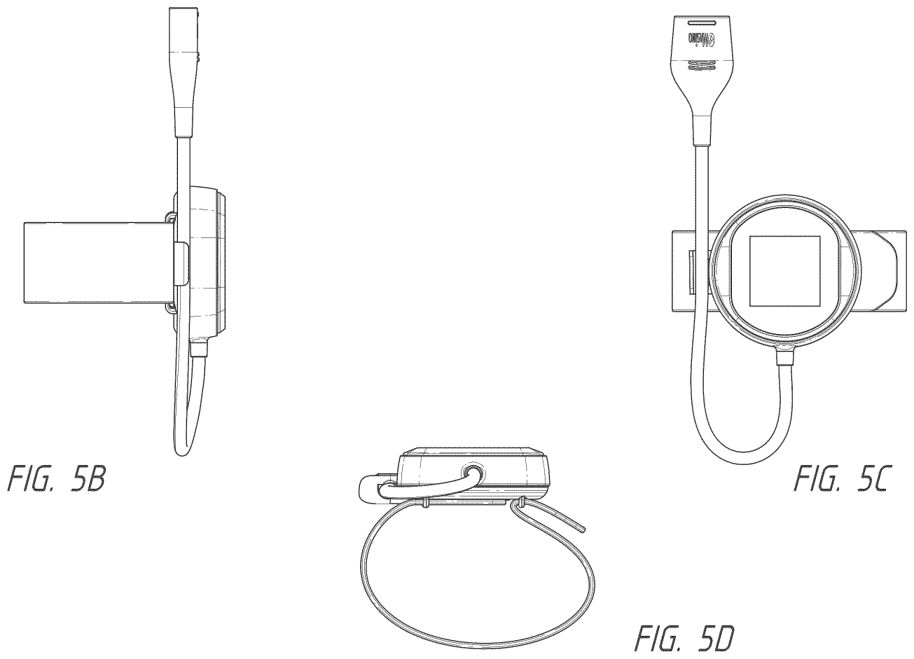

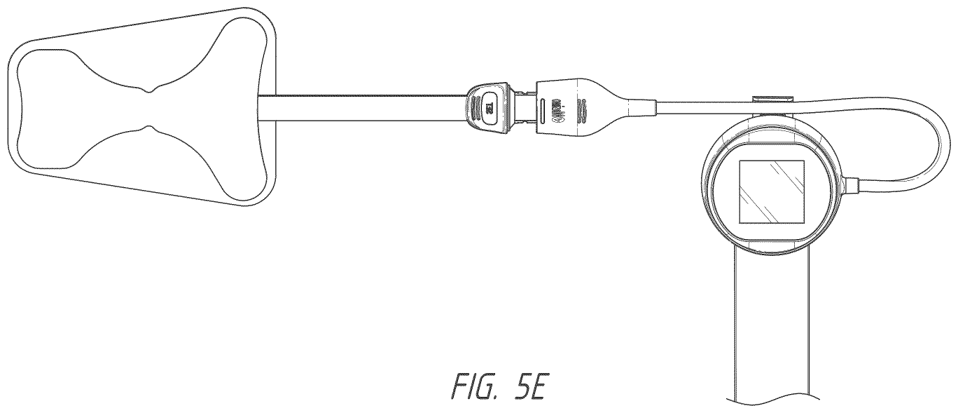

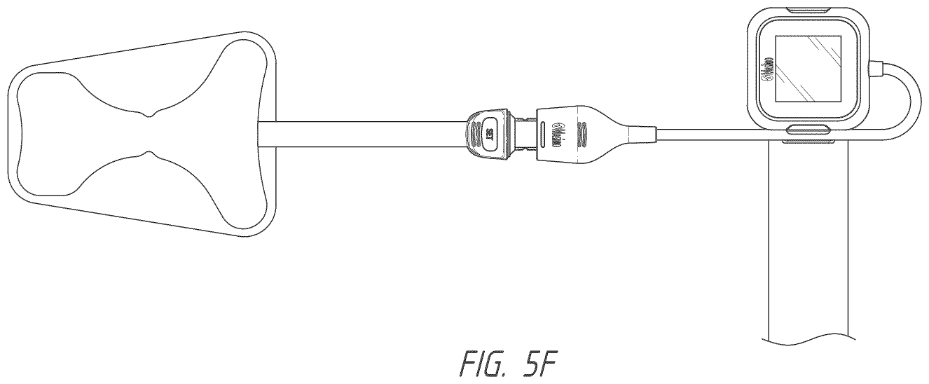

As shown in FIGS. 2A-B, the monitor instrument 110 can be detachable from the base 140. The monitor instrument 110 can have a substantially rectangular shape with an anterior surface 112 and a posterior surface 114. The anterior surface 112 faces away from the base 140. The posterior surface 114 faces toward the base 140. In some embodiments, the monitor instrument 110 can have a length of about 50-70 cm. In some embodiments, the monitor instrument 110 can have a width of about 40-60 cm. The anterior and posterior surfaces 112, 114 can be substantially flat and have a small thickness between the anterior and posterior surfaces 112, 114. The shape, size, and/or weight of the monitor instrument 110 can advantageously resemble a shape size, and/or weight of a watch and be suitable for being worn on the wrist of the patient. The shape size, and/or weight of the monitor instrument 110 are not limiting; however, in an embodiment, the size and weight are approximate that of a wrist watch. For example and not by way of limitation, the monitor instrument can have a circular outer shape as shown in FIGS. 5A-D, or a square outer shape as shown in FIG. 5F. In the illustrated embodiments, the anterior surface 112 can have a display screen 113 for displaying messages and/or physiological parameters for the patient and/or care providers.

As shown in FIG. 2A, the posterior surface 114 of the monitor instrument 110 can include a cover 116 having a group of pogo pin holes. One end of a plurality of pogo pins 117 can protrude from the pogo pin holes of the cover 116. Another end of the pogo pins can form an electrical connection with the PCB inside the monitor instrument 110 to establish an electrical connection between the PCB inside the monitor instrument 110 and one or more sensors, which will be described in more details below. More details of the pogo pins are described in U.S. application Ser. No. 15/017,349 referenced above. In the illustrated embodiment, the plurality of pogo pins 117 is arranged in two rows. A person of ordinary skill in the art will appreciate from the disclosure herein that the configuration of the plurality of pogo pins is not limiting. Additionally, although FIG. 2A shows one cover 116 with a plurality of pogo pins 117 in a center of the posterior surface 114, the number and/or locations of covers with a plurality of pogo pins are not limiting. For example and not by way of limitation, the posterior surface 114 of the monitor instrument 110 can have two covers with pogo pins on opposite ends of the posterior surface 114 along its length or width. In another example, the posterior surface 114 of the monitor instrument 110 can have one cover with pogo pins on each of four corners of the substantially rectangular posterior surface 114.

With continued reference to FIGS. 2A-2B, the base 140 can be made from disposable material(s). Disposability advantageously provides a more sterile environment for patients. That is, in an embodiment, the portions of the device that can come in contact with a patient, such as sensors 170, 172, the strap 160, and the base 140, can be single use items, while the relatively expensive processing components of the monitor instrument 110 can be sanitized, sterilized or the like, and reused. For example and not by way of limitation, the base 140 can be made from plastic materials. The base 140 can have an outer shape corresponding to the outer shape of the monitor instrument 110. As shown in FIGS. 2A-B, the base 140 has a substantially rectangular shape with an anterior surface 142 and a posterior surface 144 (shown in FIG. 2F). The anterior surface 142 faces toward the monitor instrument 110. The posterior surface 144 faces away from the monitor instrument 110 and toward the patient wearing the device. The anterior 142 and posterior 144 surfaces can be substantially flat and have a small thickness between the anterior 142 and posterior 144 surfaces. The shape and size of the base 140 are not limiting. The anterior surface 142 of the base 140 can have a recessed flat surface 143 configured to accommodate the posterior surface 114 of the monitor instrument 110. As shown in FIGS. 1A-B and 2A-B, the monitor instrument 110 can removably engage the anterior surface 142 of the base 140. In the illustrated embodiment, the base 140 can have two tabs 148 configured to clip onto or otherwise mechanically and removably mate with two recesses 118 on the monitor instrument 110. The tab 148 can have a protrusion 149 configured to fit into an indent 119 on the recess 118 of the monitor instrument 110. Other methods of removably coupling the monitor instrument 110 and the base 140 can include a magnet, a clip, a band, a snap fit, a friction fit, twist and secure, slide and secure, or otherwise, and are not limiting.

The base 140 can include one or more outlets for accommodating one or more sensor cables extending out of and away from the base 140. As shown in FIGS. 1A-C and 2A-B, the base 140 can include a first outlet 150 on a first end of the base 140 and a second outlet 152 on a second end of the base 140. In the illustrated embodiment, the second end can be opposite the first end along a length of the base 140. A first cable 174 of first sensor 170 can extend away from the base 140 via the first outlet 150. A second cable 176 of a second sensor 172 can extend away from the base 140 via the second outlet 152. Disposing outlets on opposite ends of the base 140 can advantageously prevent cluttering and tangling of the sensor cables. In the illustrated embodiment, the first sensor 170 can comprise an SpO₂ sensor and the second sensor 172 can comprise a respiratory rate sensor. Types of sensor that can connect to the base 140 are not limiting. In some embodiments, the base 140 can include only one outlet configured for any type of physiological sensor. In some embodiments, the cable(s) of the one or two sensors can be permanently connected to the outlets of the base. The base and the sensors can be both disposable. As shown in FIGS. 1B and 1C, locations of the first and second sensors 170, 172 can be exchangeable so that the first sensor 170 is connected from the side of the second outlet 152 and the second sensor 172 is connected from the side of the first outlet 150.

FIGS. 1D-1I illustrate embodiments of the wireless patient monitoring device 10 having the first and second outlets 150, 152 on the same end of the base 140. Some or all of remaining features of the wireless patient monitoring device 10 in FIG. 1D-1I can have the same structural details as the wireless patient monitoring device described above. In addition, features of the patient monitoring device 10 in FIGS. 1D-1I can be incorporated into features of patient monitoring device illustrated in the subsequent figures and described below and features of the patient monitoring device illustrated in the subsequent figures and described below can be incorporated into features of patient monitoring device 10 as illustrated in FIGS. 1D-1I. In these embodiments, the first and second cables 174, 176 of the first and second sensors 170, 172, respectively, can extend from the first and second outlets, 150, 152 on the same end of the base 140. As shown in FIGS. 1D-1I, the first cable 174 can be positioned approximately 180° relative to a direction the outlet 150 faces so that when the device 10 is worn by the user, the first and second sensors 170, 172 can be located on opposite ends of the base 140. A skilled artisan will recognize that either one of the first and second cables 174, 176 can be positioned approximately 180° relative to a direction that the outlets 150, 152 face to make the first and second sensors 170, 172 on the opposite ends of the base 140. A skilled artisan will also recognize that either one or both of the first and second cables 174, 176 can be positioned in a direction about 90°, about 180°, or about 270°, or any other angles, relative to a direction that the outlets 150, 152 face, depending on the desired locations of the sensors. A skilled artisan will appreciate from the disclosure herein that one or more outlets can be positioned anywhere along a perimeter of the wireless patient monitoring device, or on any surface of the wireless patient monitoring device, or on any surface or sides of the base 140. If two or more outlets are positioned on one side or surface of the patient monitoring device 10 or base 140, the two or more outlets can be spread out based on, for example, desired positioning of the sensors. In some embodiments, the base 140 and the one or more sensors can be unitary such that the base and the one or more sensors can be a single disposable part.

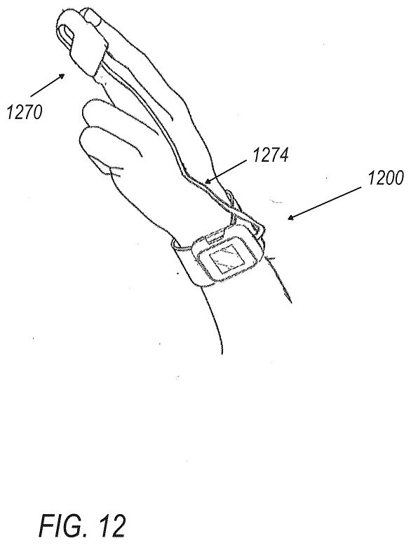

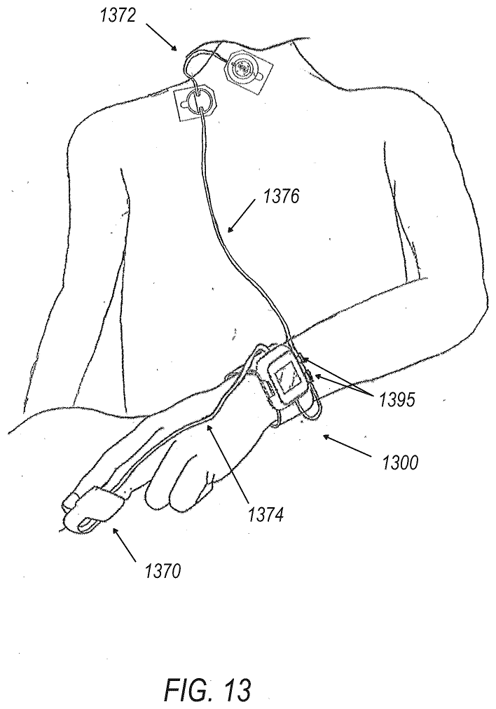

To maintain the first sensor 170 on the opposite side of the base 140 from the second sensor 172, a cable management system, for example, a cord snapping feature 195 can be used to retain the cable 174 after it is positioned approximately 180° relative to the direction the outlet 150 faces. In the illustrated embodiment, the cable management system 195 can retain a portion of the first cable 174 by a snap fit, although methods of retaining the cable are not limiting. In addition to maintaining the position of the first sensor 170, the cable management system 195 can allow a length of the first cable 174 relative to the base 140 to be adjusted to prevent the first cable 174 from dangling about the patient's wrist or arm. A skilled artisan will recognize from the disclosure herein a wide range of mechanical mating or other mechanisms for positioning and managing the positions of the cables. FIG. 12 illustrates a patient wearing an example wireless patient monitoring device 1200 on the patient's wrist. In the illustrated embodiment, the device 1200 is connected to one sensor 1270. The sensor 1270 can be a pulse oximeter sensor and the patient can wear the sensor 1270 on the patient's fingertip, with the sensor cable or flex-circuit 1274 extending between the device 1200 and the sensor 1270. The device 1200 can include a cable management system described herein to retain a portion of the cable or flex-circuit 1274 and allow a length of the cable or flex-circuit 1274 to be adjustable. FIG. 13 illustrates a patient wearing an example wireless patient monitoring device 1300 on the patient's wrist. In the illustrated embodiment, the device 1300 is connected to a first sensor 1370 and a second sensor 1372. The first sensor 1370 can be a pulse oximeter sensor and the patient can wear the first sensor 1370 on the patient's fingertip. The second sensor 1372 can be an acoustic sensor and the patient can wear the second sensor 1372 near or around the patient's neck. As shown in FIG. 13, a cable management system 1395 can retain a portion of the sensor cable 1376 connecting the second sensor 1372 and the device 1300 and allow a length of the cable 1376 to be adjustable. The device 1300 can further include a cable management system described herein to retain a portion of the cable or flex-circuit 1374 connecting the first sensor 1370 and the device 1300, and allow a length of the cable or flex-circuit 1374 to be adjustable.

As shown in FIGS. 1D-1F, the cable management system 195 can be a slidable cord-snap component configured to slide along the first cable 174 and be snapped onto a slot 196 on the monitor instrument 110 to retain the first cable 174 relative to the monitor instrument 110. As shown in FIGS. 1G-1H, the cable management system 195 can be one or more cord snap features attached to, or be an integral part of the monitor instrument 110 or the base 140. In some embodiments, two or more cable management systems 195 can be located on the monitor instrument 110 or the base 140 to retain the first cable 174. In some embodiments, additional cable management systems can be available to retain both of the first and second cables 174, 176 and make the cable lengths between the patient monitoring device 10 and both the first and second sensors 170, 172 adjustable. A skilled artisan will recognize that the cable management systems can be located on any suitable locations of the wireless patient monitoring device 10.

Electrical connections of the sensor(s) to the monitor instrument 110 will now be described. With continued reference to FIGS. 2A-2B, the anterior surface 142 of the base 140 can include a pad 146 having a plurality of electrical contacts 147 on one side of the pad 146. The pad 146 can be a PCB. In some embodiments, the pad 146 can have one or more EEPROMs or other electronic components. Each EEPROM can store identification information of a sensor, schemes for validating the authenticity of the sensor, and other information relating to the sensor. The one or more EEPROMs or other electronic components can be on the same side or reverse side of the pad 146 that has the plurality of electrical contacts 147. FIGS. 2D-E illustrate some non-limiting examples of the pads. The pad 146 can be molded onto the anterior surface 142 of the base 140. The pad 146 can be disposable with the rest of the base 140. The electrical contacts 147 can be electrically connected to at least one electrical connector. The electrical connector(s) can include electronics configured for connecting to one or more of the sensors 170, 172. Specifically, the electrical contacts 147 can be electrically connected to the cables 174, 176 by soldering one or more wires of each cable to a group of soldering points on the pad 146. The group of soldering points can be on the same side or reverse side of the pad 146 that has the plurality of electrical contacts 147. Thus, the PCB advantageously facilitates electrical communication between conductors of the cables 174, 174 and the processing device(s) of the instrument 110. Specifically, in an embodiment, the processor communicates with pogo style electrical pins housed in the instrument 110. When seated or otherwise fixed to the base 140, the pogo pins form an electrical connection with the electrical contacts 147. The electrical contacts 147 are in electrical communication with soldering points 254, 256 (shown in FIG. 2E), and in some embodiments, one or more information elements like an EEPROM, which are in turn in electrical communication with conductors of one or more of the cables 174, 176. In an embodiment, this electrical pathway electrically bridges the instrument 110 to one or more of the sensors through the base 140.

FIG. 2D shows a pad 200 having one group of soldering points 204 on a first side 208 of the pad 200. The pad 200 can have a second side 212 opposite the first side 208 The second side 212 can include a plurality of electrical contacts 216 configured to contact the pins 117, for example, as shown in FIG. 2A. The second side 212 can have one or more EEPROMs or other electronic components 220. The plurality of electrical contacts 216 can be on a recessed surface due to a thickness of the one or more EEPROMs or other electronic components 220. The pins 117 can be configured to have a length suitable for contacted the electrical contacts 216 on the recessed surface. The pins 117 and the electrical contacts 216 can be surrounded by common projections to establish electrical connection between the pins 117 and the electrical contacts 216. In some embodiments, the one or more EEPROMs or other electronic components 220 can be located on the first side 208 so that the electrical contacts 216 can be flush with a surface of the second side 212 of the pad 200. Having the electrical contacts 216 flush with the surface of the second side 212 of the pad 200 can ensure adequate contacts between the pins 117 and the electrical contacts 216. In addition, soldering of the one or more EEPROMs or other electronic components 220 and the cable wires to the pad 200 can be done on the same side of the pad 200

FIG. 2E shows another pad 250 having two groups of soldering points 254, 256 on a first side 258 of the pad 250. The two groups of soldering points 254, 256 can be configured to each accommodate wires from a sensor cable. The first side 258 can have at least two EEPROMs or other electronic components 270 located between the two groups of soldering points 254, 256. The pad 250 can have a second side 262 opposite the first side 258. The second side 262 can include a plurality of electrical contacts 266 configured to contact the pins 117, for example, as shown in FIG. 2A. The electrical contacts 266 are flush with a surface of the second side 262 of the pad. As described above, having the electrical contacts 266 flush with the surface of the second side 262 of the pad 250 can ensure adequate contacts between the pins 117 and the electrical contacts 266. One advantage of soldering two sensor cables to the pad 250 to establish electrical connection between the sensor(s) and the monitor instrument is that the cable wires can flex in all directions, making it easy to position the sensor(s) relative to the monitoring device.

In some embodiments, the electrical connection of the sensors and the monitor instrument can include a hybrid connector to accommodate one sensor cable and one flex-circuit. One of the sensors, such as the sensor 170, can include a flex-circuit instead of being connected to conducting wires of a sensor cable. The plurality of electrical contacts for contacting the pins can be located on or an integral part of the flex circuit, which incorporates, for example, conductive traces instead of conductive wires. The flex circuit can include a stiffening part, such as a flat board, behind the electrical contacts. Stiffening the electrical contacts portion of the flex circuit can increase the rigidity of the electrical contacts, thereby ensuring adequate contact between the pins and the electrical contacts. The flex-circuit can include an extension having a group of soldering points. Cable wires of the sensor cable for connecting to a second sensor, such as the sensor 172, can be soldered onto the group of soldering points. The extension can optionally be supported by a stiffening board. Because of the flexibility of the flex-circuit, the extension having the group of soldering points can be folded under the electrical contacts or at other locations to expose the electrical contacts for contacting the pins. Additional details of the flex-circuit are described in U.S. application Ser. No. 13/951,313, filed on Jul. 25, 2013 and entitled “AUTOMATED ASSEMBLY SENSOR CABLE,” which is expressly bodily incorporated in its entirety and is part of this disclosure. An artisan will recognize from the disclosure herein that one or more cables, individual cables, or all cables could advantageously include one or more flex circuits.

In the illustrated embodiment, the plurality of electrical contacts 147 can be arranged in two rows and located in a center of the anterior surface 142 of the base 140 so as to be able to overlap with the pad 116 on the posterior surface 114 of the monitor instrument 110 as shown in FIG. 2A. One of ordinary skill in the art will appreciate from the disclosure herein that the number and arrangement of the pad 146 with the plurality of electrical contacts 147 are not limiting. For example and not by way of limiting, the anterior surface 142 of the base 140 can have four pads 146 with a plurality of electrical contacts 147, one on each corner of the substantially rectangular anterior surface 142 of the base 140, and the posterior surface 114 of the monitor instrument 110 can have four corresponding covers 116 with a plurality of pogo pins 117 on the four corners of the posterior surface 114 of the monitor instrument 110.

As described above, the cables 174, 176 can extend outside the base 140 at the outlets 150, 152, respectively. In some embodiments, the outlets 150, 152 can include the electrical connectors, such as mechanical plugs that are electrically connected to the electrical contacts 147. The first and second sensor cables 174, 176 can be plugged into the mechanical plugs. In some embodiments, the mechanical plug can include a phone plug or the like. Although two separate outlets are shown in the illustrative example, the wireless patient monitoring device 10 can include a single outlet with two plugs, or a multi-port connector configured for connecting to a plurality of sensors of different types and/or sizes.

When the monitor instrument 110 is removably engaged with the base 140, the posterior surface 114 of the monitor instrument 110 can overlap with the anterior surface 142 of the base 140. The pogo pins 117 on the monitor instrument 110 can come into contact with the electrical contacts 147 on the base 140, thereby establishing electrical connections between the printed circuit boards inside the monitor instrument 110 and the sensors 170, 172. In some embodiments, when the posterior surface 114 of the monitor instrument 110 comes into close proximity with the anterior surface 142 of the base 140, the pogo pins 117 can retract into the pogo pin holes while still maintaining electrical connection with the electrical contacts 147. The electrical connection between the monitor instrument 110 and the sensors 170, 172 can allow the sensors 170, 172 connected to the base 140 to communicate with and send sensor data to the monitor instrument 110. Having the electrical contacts for the pogo pins on the base can advantageously reduce a size of a connector between a sensor and a monitor, or between a sensor and a sensor cable, and make the connecting structures less bulky. The less bulky connecting structures can advantageously provide more comfort to the patient. One of ordinary skill in the art will also appreciate from the disclosure herein that types of electrical connectors other than pogo pin connectors can be used to electrically connect monitor instrument 110 and the base 140.

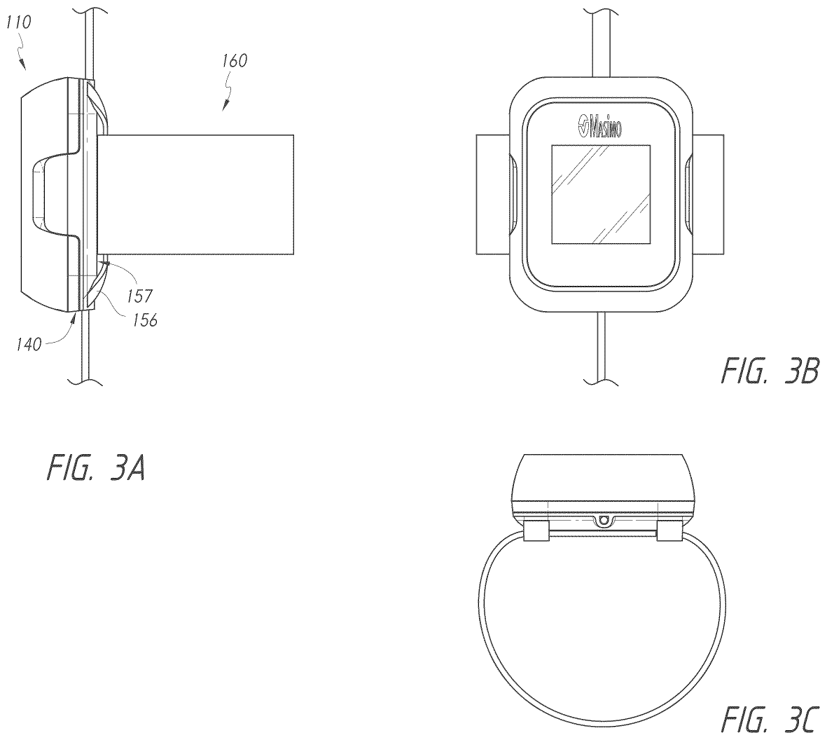

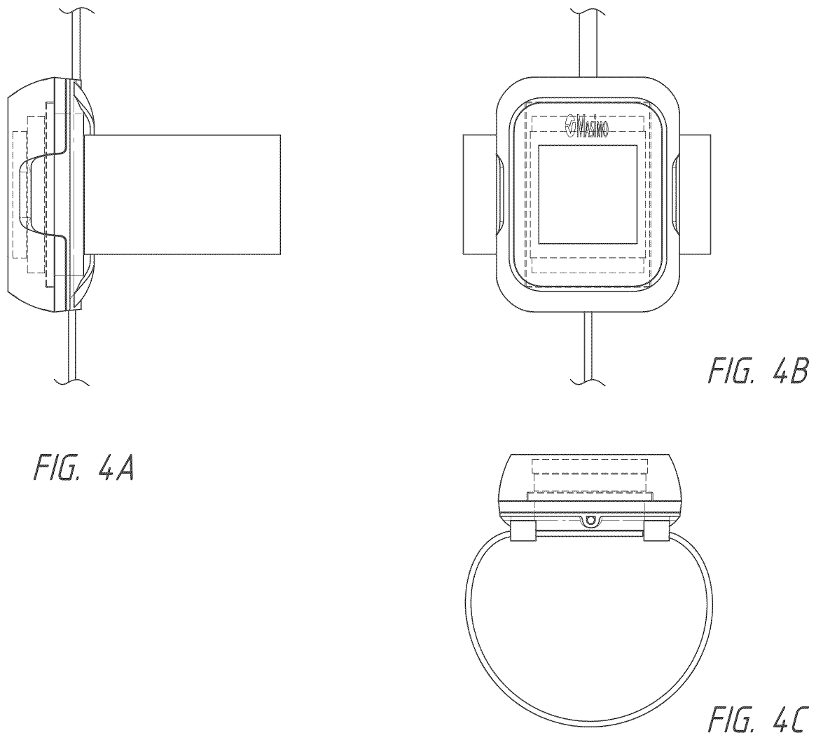

As shown in FIGS. 1F, 2F, and 3A, the base 140 can include one or more strap connectors 156 for engaging the strap 160. The strap connector 156 can be an integral portion of the base 140 or a separately formed component secured to the base 140 mechanically, or via adhesives or welding, or the like. The strap connector 156 can form an opening 157 with the posterior surface of the base 140. The strap 160 can pass through the opening 157 to be secured to the base 140. As shown in FIGS. 1F and 2F, the base 140 can have two strap connectors 156 on opposite ends across a width of the base 140.

The strap 160 can include any fabric, elastic, or otherwise flexible material. In certain embodiments, the strap 160 can be waterproof. One or both ends of the strap 160 can be tapered. One or both ends of the strap 160 can include a covering to protect the strap ends. The strap 160 can be secured to the patient's wrist as a wristband, or in any other configuration. A portion of the strap 160 can be secured to another portion of the strap 160 using Velcro, clasps, adhesive, snap-fits, or any other connector. The strap 160 can include any or all of the features of the strap described in U.S. application Ser. No. 13/762,270, filed Feb. 7, 2013, titled “Wireless Patient Monitoring Device,” the disclosure of which is hereby incorporated by reference in its entirety. In an embodiment, the strap can include a foam or posy wrap type material common in securing mechanisms for patient sensor, such as neonate or infant sensors. Each physiological sensor, such as one of the sensors 170, 172, can include its own sensor attachment mechanism separate from the base 140 and the strap 160. The sensor attachment mechanism can be configured to removably secure the physiological sensor to a measurement site on the patient. Each sensor can include a sensor positioner configured to position the sensor with respect to the measurement site on the patient. In an embodiment, the sensor attaches using an adhesive layer. Other embodiments will be known to an artisan from the disclosure herein, including, for example, a Posey wrap, Velcro, tape, mechanical couplings generally having a closed bias to grip or otherwise stick to a measurement site, or other commercially available attachments.

Providing the patient monitoring device 10 wearable on the wrist can advantageously allow the patient to easily check the patient's physiological state or parameters by looking at the display screen of the monitor. Other advantages of the wearable patient monitoring device 10 include reducing clutter of cables, improving patient mobility by eliminating some or all of the cables.

In some embodiments, the patient monitoring device can removably connect to a sensor via a sensor cable connector. Examples of such patient monitoring devices are shown in FIGS. 5A-11D. In these embodiments, the sensor cable connector can extend from the reusable monitor instrument and the disposable base can include no electrical components. As shown in FIGS. 5A-7E, the patient monitoring device 50 can have features of the patient monitoring device 10 of FIGS. 1A-2B except as described below. Accordingly, features of the patient monitoring device 50 can be incorporated into features of patient monitoring device 10 and features of the patient monitoring device 10 can be incorporated into features of patient monitoring device 50. The monitor instrument 510, the base 540, and the strap 560 can operate in the same or similar manner to the operation of the monitor instrument 110, the base 140, and the strap 160 described above.

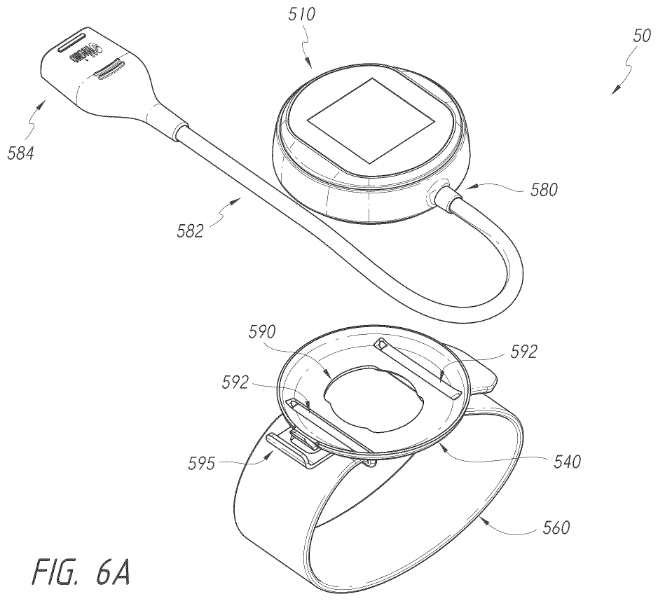

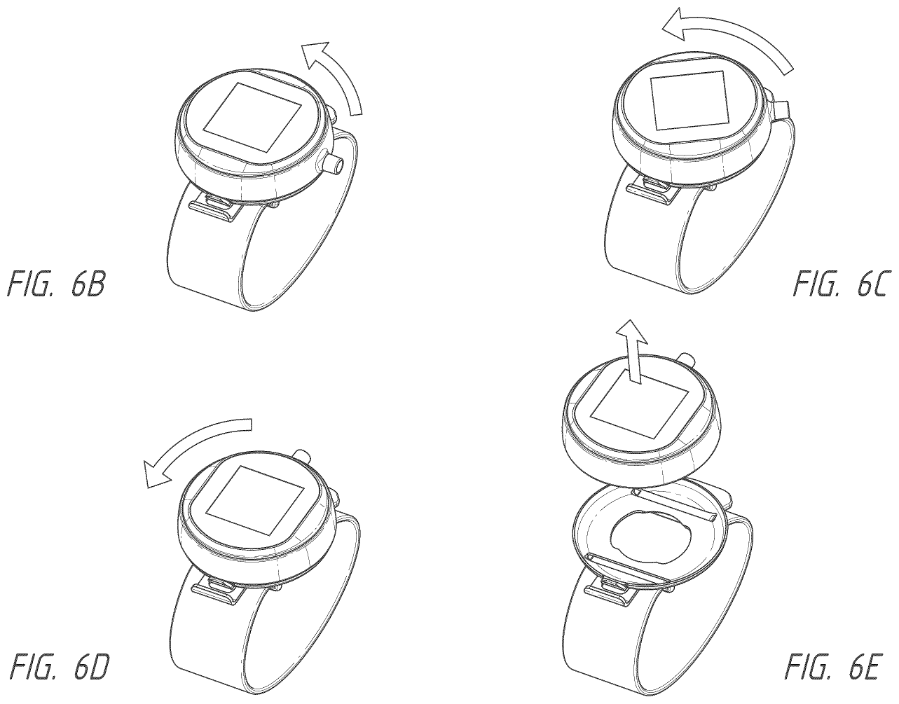

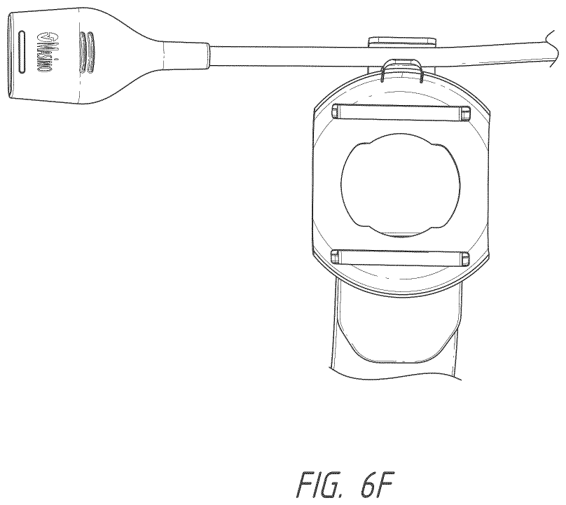

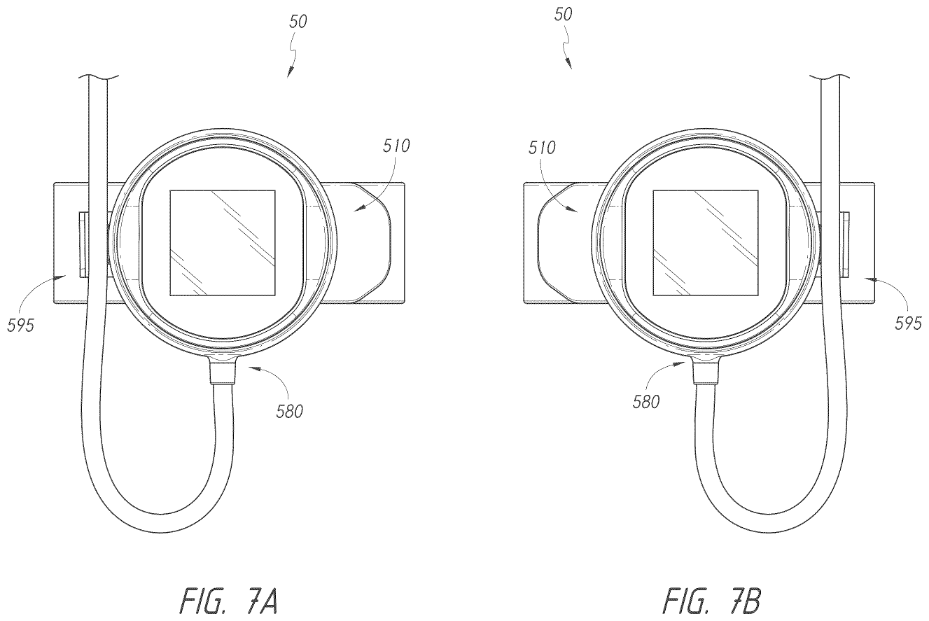

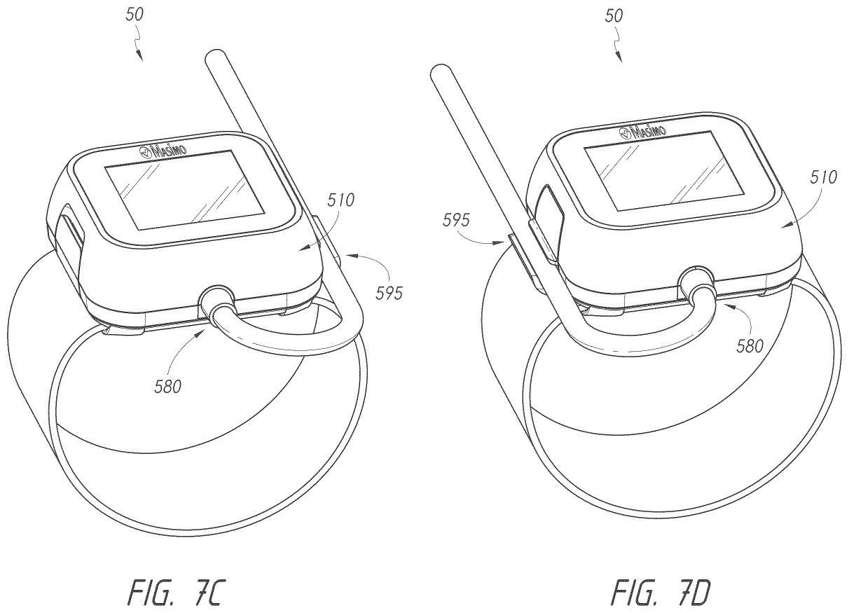

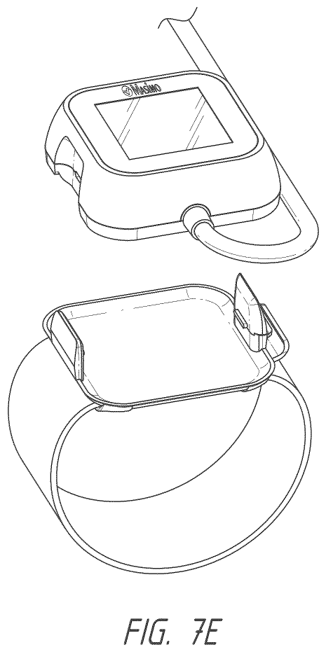

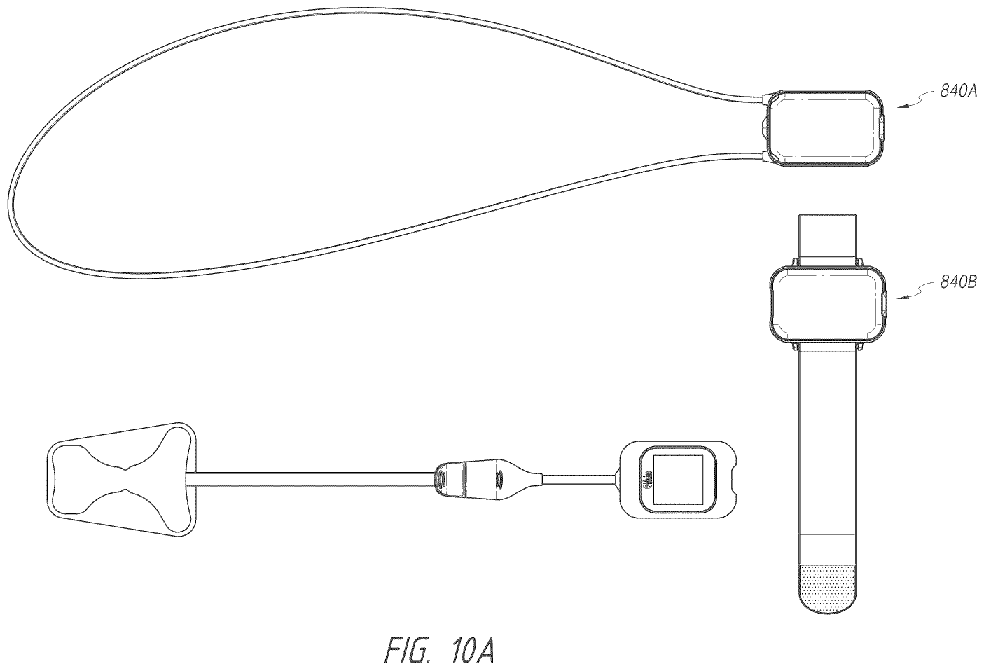

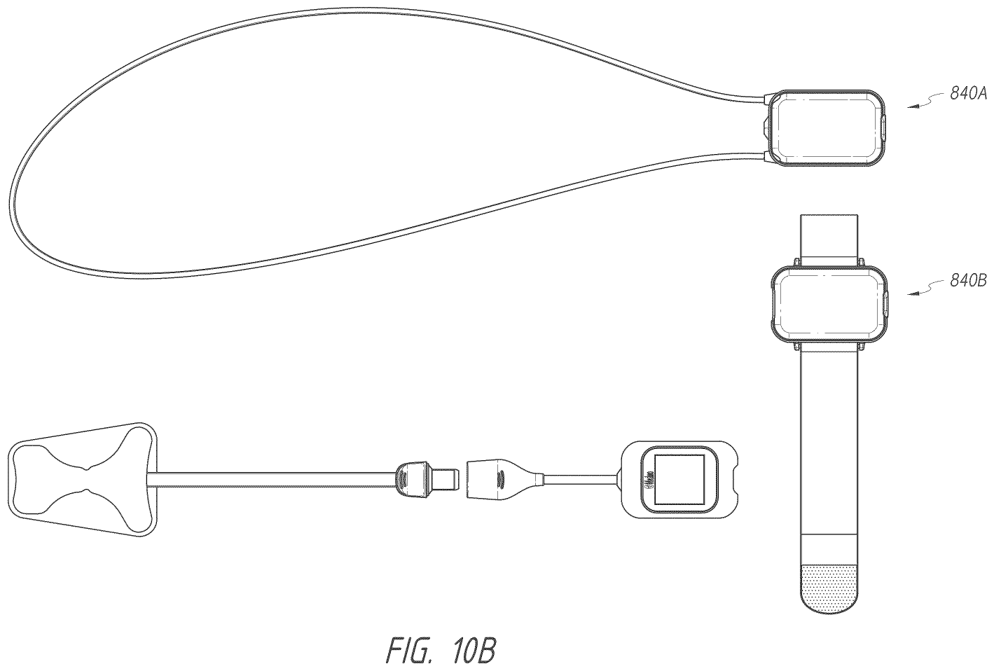

As shown in FIGS. 5A and 6A, the monitor instrument 510 and the base 540 can both have round outer shapes. The base can have a corresponding round outer shape. In some embodiments, such as shown in FIG. 6F, the base can have a corresponding round outer shape with two flat sides along a length of the strap. The two flat sides can reduce a foot print of the base when the device is worn by the patient, thereby making the device more comfortable to wear. In other embodiments, such as shown in FIGS. 5F and 7E, the monitor instrument 510 and the base 540 can have a square or rectangular outer shape. The monitor instrument 510 can have a cable outlet 580 on a side wall of the monitor instrument 510. A sensor connector cable 582 can extend from the cable outlet 580. In some embodiments, the sensor connector cable 582 can be permanently coupled to the cable outlet 580. The sensor connector cable 582 can be electrically connected to an electrical circuit in the monitor instrument 510. The sensor connector cable 582 can terminate on a free end at a sensor cable connector 584. In some embodiments, the sensor cable connector 584 can comprise pogo pin connectors. Types and methods of electrically connecting the sensor cable connector 584 and a sensor are not limiting. A sensor (shown in FIGS. 10A-B) removably connected to the sensor cable connector 584 can send sensor data to the monitor instrument 510.

Also as shown in FIGS. 5A and 6A, the base 540 can have an opening 590 for engaging, and mechanically and removably mating with a complementary protruding portion on the posterior surface of the monitor instrument 510. The opening 590 can have an irregular shape configured for rotationally retaining the monitor instrument 510. In the illustrated embodiment, the opening 590 can have an outer shape of two substantially rectangular shapes overlapping with each other, one of the substantially rectangular shapes being generally perpendicular with the other one of the substantially rectangular shapes. The base 540 can optionally have one or more open slots 592 to aid the positioning and engagement between the base 540 and the monitor instrument 510. The complementary protruding portion on the monitor instrument 510 can pass through the opening 590 when a length of the protruding portion aligns with the length of the open 590 and a width of the protruding portion aligns with the width of the opening 590. The monitor instrument 510 can then be turned clockwise or anticlockwise about a quarter of a turn to secure the monitor instrument 510 to the base 540. As shown in FIG. 5A, when the monitor instrument 510 is engaged with the base 540, the cable outlet 580 can be pointing away from the strap 560 and substantially parallel to a width of the strap 560. This configuration of the cable outlet 580 can advantageously prevent the sensor connector cable 582 from contacting the strap 560 near the cable outlet 580, which can cause stress to and early failure of the sensor connector cable 582. This configuration can also allow the patient's wrist to move freely without being hindered by the sensor connector cable 582 extending from the cable outlet 580. FIGS. 6B-E illustrate reverse steps for removing the monitor instrument 510 from the base 540, such as by rotating the monitor instrument 510 anticlockwise or clockwise about a quarter of a turn so that a length of the protruding portion can align with the length of the open 590 and a width of the protruding portion can align with the width of the opening 590.

With continued reference to FIGS. 5A-6E, the base 540 can have a cord snap feature 595 similar to the cord snap feature 195 described above. The cord snap feature can be on a circumference of the base 540. The cord snap feature 595 can retain a portion of the sensor connector cable 582 and prevent the sensor connector cable 582 from dangling about the patient's wrist or arm. In the illustrated embodiment, the cord snap feature 595 can retain a portion of the sensor connector cable 582 by a snap fit, although methods of retaining the sensor connector cable 582 are not limiting. As shown in FIGS. 5A-6E, the cord snap feature 595 can be located along the width of the strap 560. The cord snap feature 595 can also be located substantially 90° from the cable outlet 580 when the monitor instrument 510 engages the base 540. The configuration of the cord snap feature 595 can advantageously allow the sensor connector cable 582 to be snapped on the cord snap feature 595 without having to make sharp turns. The configuration of the cord snap feature 595 can also advantageously allow the sensor connector cable 582 to run substantially parallel to the patient's arm when the patient wears the patient monitoring device 50 on her wrist.

As shown in FIGS. 7A-E, the cord snap feature 595 can be about 90° clockwise from the cable outlet 580 or about 90° counterclockwise from the cable outlet 580 when the monitor instrument 510 engages the base 540. These alternative configurations of the cord snap feature 595 can advantageously aid in the ergonomics of the device and cable management, and can accommodate both patients who prefer to wear the monitoring device 50 on the left wrist and patients who prefer to wear the monitoring device 50 on the right wrist. However, an artisan will recognize from the disclosure herein that the snap feature 595 can be in virtually any position with respect to the outlet 580 that provides for reduced clutter, better positioning of the sensor, reduced mechanical stress on the cable or cable connectors, or reduces pinching of the cable, or any other advantageous.

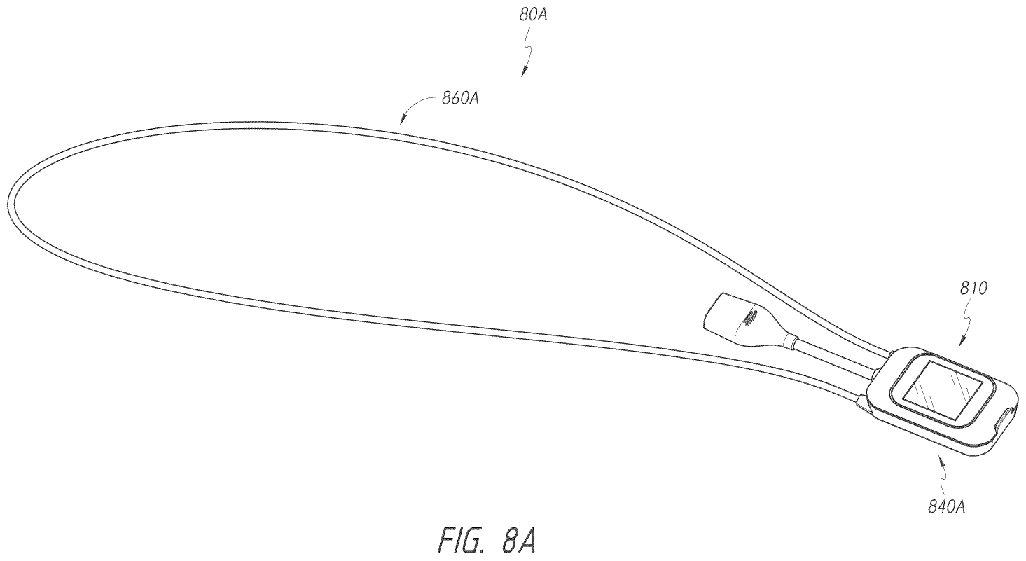

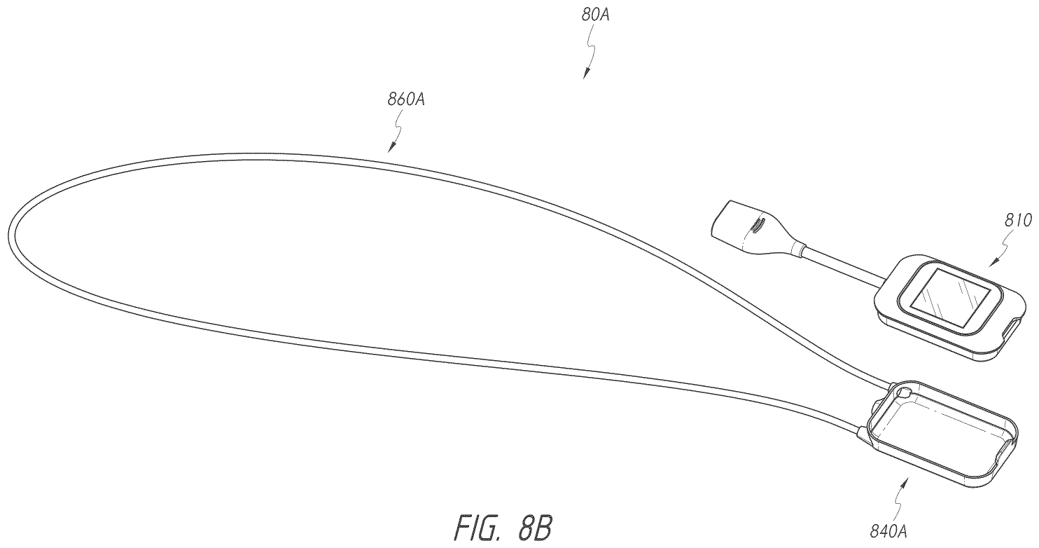

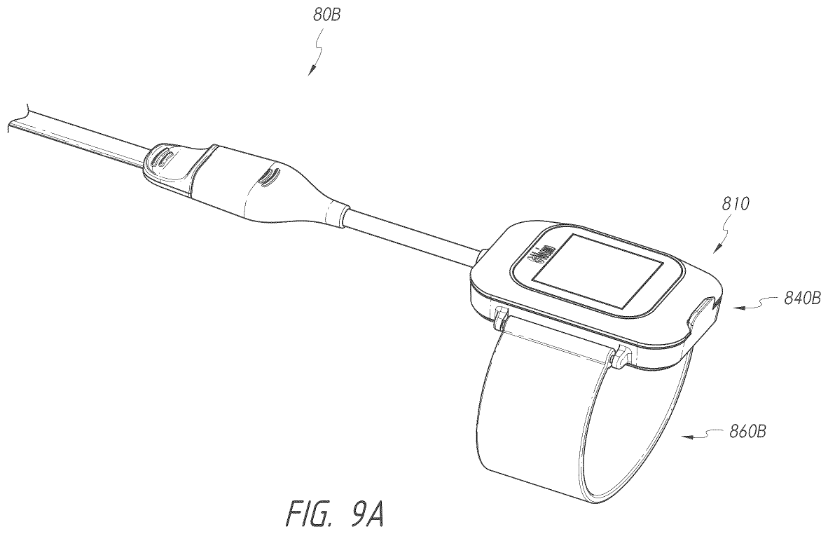

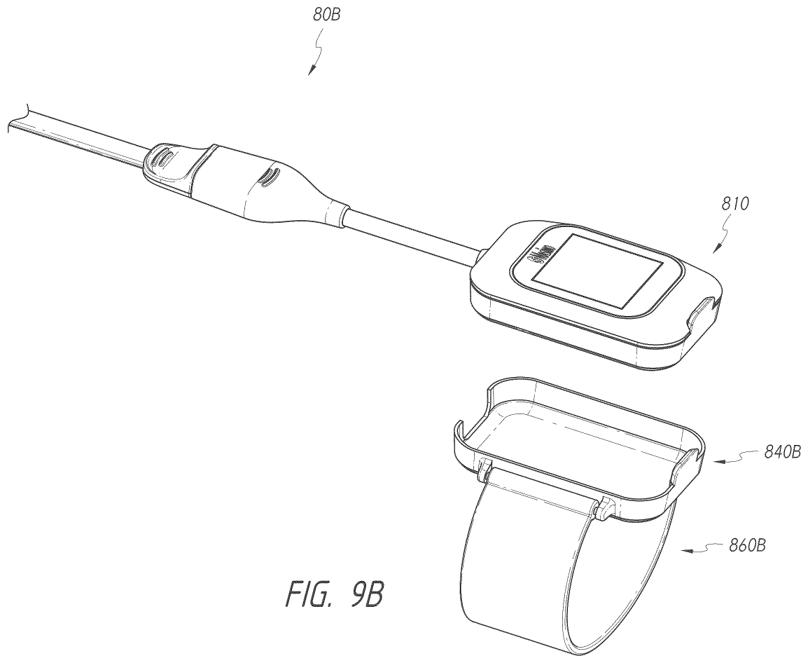

FIGS. 8A-9B illustrate embodiments of the patient monitoring device 80A, 80B. The patient monitoring devices 80A, 80B can have features of the patient monitoring device 50 except as described below. Accordingly, features of the patient monitoring devices 80A, 80B can be incorporated into features of patient monitoring device 50 and features of the patient monitoring device 50 can be incorporated into features of patient monitoring devices 80A, 80B. The monitor instrument 810, the bases 840B, and the strap 560B as shown in FIGS. 9A-B can operate in the same or similar manner to the operation of the monitor instrument 510, the base 540, and the strap 560 described above. The monitor instrument 810 can be configured to be compatible with both the bases 840A, 840B such that the patient can choose between wearing the device 80A around the neck, or wearing the device 80B on a wrist or arm.

As shown in FIGS. 8A-B, the base 840A of the patient monitoring device 80A can be connected to a cord 860A instead of the strap 860B. The cord 860A can be worn around the patient's neck. The cord 860A can advantageously allow the patient monitoring device 80A to be coupled with an in-ear and/or nose sensor (not shown) without requiring a long cable connecting the in-ear and/or nose sensor and the base 860A. Although the cord is described in connection with embodiments of the monitor instrument including a sensor cable connector, the cord can also be incorporated into embodiments of the patient monitoring device 10 described above such that the base 140 can be connected to a cord instead of being connected to the strap 160.

As shown in FIG. 10A-B, the bases 840A, 840B can both be compatible with the monitor instrument 810. For example, the bases 840A, 840B can have the same coupling features for engaging the monitor instrument 810 as described above. Accordingly, the same monitor instrument 810 can removably engage either the base 840B for wearing the patient monitoring device 80B on the wrist or the base 840A for wearing the patient monitoring device 80A around the neck. Interchangeability between the bases 840A, 840B can advantageously allow the monitor instrument 810 to be used with various types of the sensors depending on where the sensors need to be located on the patient's body.

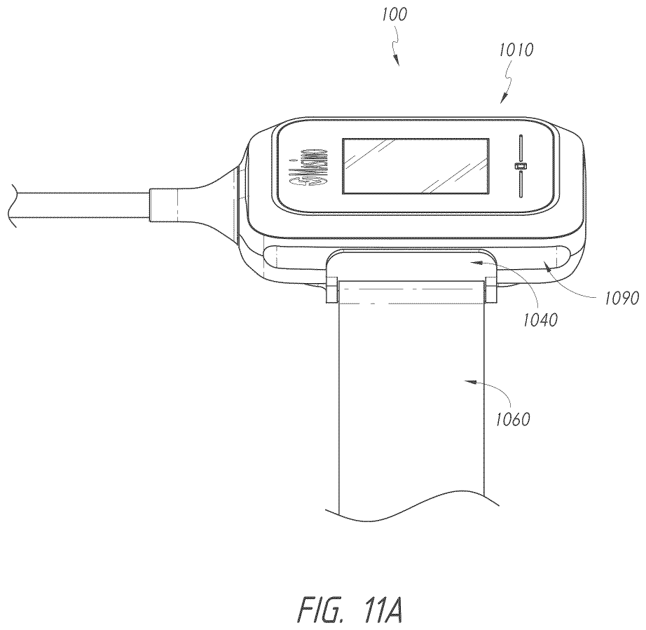

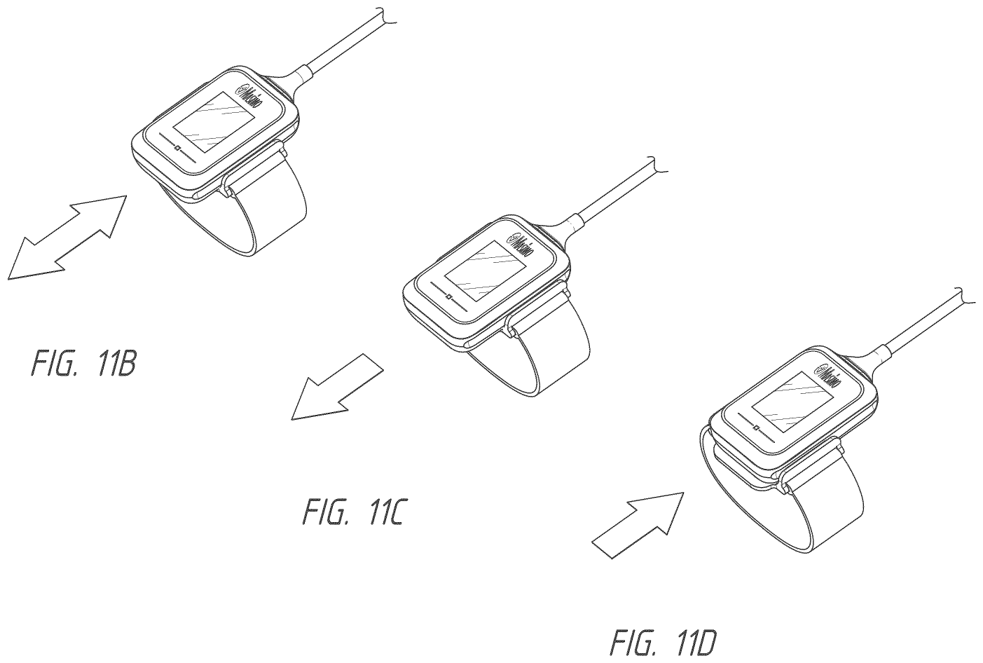

Turning to FIGS. 11A-D, another embodiment of the patient monitoring device 100 is shown. The patient monitoring device 100 can have features of the patient monitoring device 50 except as described below. Accordingly, features of the patient monitoring device 100 can be incorporated into features of patient monitoring device 50 and features of the patient monitoring device 50 can be incorporated into features of patient monitoring device 100. The monitor instrument 1010, the bases 1040, and the strap 1060 as shown in FIGS. 11A-D can operate in the same or similar manner to the operation of the monitor instrument 510, the base 540, and the strap 560 described above.

As shown in FIG. 11A, the monitor instrument 1010 of the patient monitoring device 100 can have four sides. There can be two sliding channels 1090 on two opposing sides. In the illustrated embodiment, the sliding channels 1090 can be located on the sides that do not have a cable outlet or other types of connection features. The base 1040 can have corresponding protrusions (not shown) along two opposing sides of the base 1040. The sliding channels 1090 can accommodate the protrusions on the base 1040 so that the monitor instrument 1010 can slide onto the base 1040. FIGS. 11B-D show that the monitor instrument 1010 and the base 1040 can slide relative to each other in two directions as indicated by the arrows. In some embodiments, the sliding channels 1090 and the protrusions can have a friction fit or other types of tolerances so that the monitor instrument 1010 stays on the base 1040 without an external force along the directions of sliding shown in FIGS. 11B-D. This sliding configuration can advantageously prevent inadvertent rotation of the monitor instrument 1010 during use. In some embodiments, the protrusions on the base 1040 can be snap-fitted into the sliding channels and the sliding channels 1090 can have two closed ends to prevent the protrusions on the base 1040 from disengaging the sliding channels 1090. The protrusions can be configured to slide along the sliding channels 1090 during use such that when the patient rotates her wrist or arm, the monitor instrument 1010 can slide back and forth along the sliding channels. The slidable monitor instrument 1010 can increase the ergonomics of the device. A skilled artisan will recognize from the disclosure herein that other types of sliding mechanisms can be used, such as a sliding rail/channel on the monitor instrument 1010 or the base 1040 with two closed ends and one or more corresponding mushroom tabs on the base 1040 or the monitor instrument 1010.

Although this disclosure has been disclosed in the context of certain preferred embodiments and examples, it will be understood by those skilled in the art that the present disclosure extends beyond the specifically disclosed embodiments to other alternative embodiments and/or uses of the disclosure and obvious modifications and equivalents thereof. In addition, while a number of variations of the disclosure have been shown and described in detail, other modifications, which are within the scope of this disclosure, will be readily apparent to those of skill in the art based upon this disclosure. It is also contemplated that various combinations or sub-combinations of the specific features and aspects of the embodiments may be made and still fall within the scope of the disclosure. Accordingly, it should be understood that various features and aspects of the disclosed embodiments can be combined with or substituted for one another in order to form varying modes of the disclosed.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. Additionally, as used herein, “gradually” has its ordinary meaning (e.g., differs from a non-continuous, such as a step-like, change).

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive. For example, the scope of the present disclosure is not limited to parameters measurable by a pulse oximeter sensor and an acoustic sensor. The wireless patient monitoring system described herein can include sensor additions or substitutions to these sensors. The sensor additions or substitutions can be configured to monitor one or more of capnography, blood pressure, ECG, EEG, electrolytes, brain function/activity, patient turning, patient fall detection, patient location, and the like. The wireless patient monitoring system can also output to a multi-parameter monitor, or a regular patient monitor, or be configured to control signals for other devices, such as infusion pumps, oxygen supply, respiratory apparatuses, and the like. Connection between the wireless patient monitoring system and the multi-parameter monitor, regular patient monitor, or other devices can be via cable, via wireless technology, or both.

What is claimed is:

1. A system for monitoring physiological information of a subject, the system comprising:
a wearable assembly configured to be secured to the subject, the wearable assembly comprising:
a strap configured to be secured to a wrist of the subject;
a base connected to the strap, the base comprising:
a bottom surface configured to face toward the subject's wrist and a top surface configured to face away from the subject's wrist;
a printed circuit board; and
a plurality of electrical contacts in electrical communication with the printed circuit board and arranged on or proximate to the top surface of the base;
a pulse oximetry sensor configured to be removably secured to a finger of the subject and generate one or more signals indicative of at least one physiological parameter of the subject; and
a cable comprising a first end electrically connected to the pulse oximetry sensor and a second end electrically connected to the printed circuit board of the base, the cable configured to transmit said one or more signals from said pulse oximetry sensor to said printed circuit board; and
a monitor instrument configured to removably secure to the base of the wearable assembly, the monitor instrument comprising:
a top surface and a bottom surface, the bottom surface of the monitor instrument configured to face toward the top surface of the base of the wearable assembly when the monitor instrument and base are secured to one another;
a plurality of electrical contacts arranged on or proximate to the bottom surface of the monitor instrument, the plurality of electrical contacts of the monitor instrument configured to contact the plurality of electrical contacts of the base of the wearable assembly and receive said one or more signals from the plurality of electrical contacts of the base when the monitor instrument and base are secured to one another;
a processor configured to receive said one or more signals from the plurality of electrical contacts of the monitor instrument and determine said at least one physiological parameter of the subject;
a wireless transceiver configured to transmit said determined at least one physiological parameter of the subject to an external device; and
a battery.

2. The system of claim 1, wherein the plurality of electrical contacts of the monitor instrument comprises pins.

3. The system of claim 1, wherein:
the base further comprises a plurality of tabs extending generally perpendicular relative to said top surface of the base;
the monitor instrument further comprises a plurality of recesses; and
each of the plurality of tabs of the base engage a different one of the plurality of recesses of the monitor instrument when the monitor instrument is secured to the base.

4. The system of claim 1, wherein the monitor instrument further comprises a display positioned on the top surface of the monitor instrument and configured to display information relating to said at least one physiological parameter.

5. The system of claim 1, wherein the cable is permanently connected to the base of the wearable assembly.

6. The system of claim 1, wherein the base further comprises a memory in communication with said printed circuit board and configured to store information relating to said pulse oximetry sensor.

7. A system for monitoring physiological information of a subject, the system comprising:
a wearable assembly configured to be secured to the subject, the wearable assembly comprising:
a strap configured to be secured to a wrist of the subject;
a base connected to the strap, the base comprising:
a printed circuit board; and
a plurality of electrical contacts in electrical communication with the printed circuit board; and
a pulse oximetry sensor in electrical communication with said printed circuit board and configured to generate one or more signals indicative of at least one physiological parameter of the subject; and
a monitor instrument configured to removably secure to the base of the wearable assembly, the monitor instrument comprising:
a plurality of electrical contacts configured to contact the plurality of electrical contacts of the base of the wearable assembly and receive said one or more signals from the plurality of electrical contacts of the base when the monitor instrument and base are secured to one another;
a processor configured to receive said one or more signals from the plurality of electrical contacts of the monitor instrument and determine said at least one physiological parameter of the subject;
a wireless transceiver configured to transmit said determined at least one physiological parameter of the subject to an external device; and
a battery.

8. The system of claim 7, wherein the plurality of electrical contacts of the monitor instrument comprises pins.

9. The system of claim 7, wherein:
the base further comprises:
a bottom surface configured to face toward the subject's wrist and a top surface configured to face away from the subject's wrist; and
a plurality of tabs extending generally perpendicular relative to said top surface of the base;
the monitor instrument further comprises:
a top surface and a bottom surface, the bottom surface of the monitor instrument configured to face toward the top surface of the base of the wearable assembly when the monitor instrument and base are secured to one another; and
a plurality of recesses; and
each of the plurality of tabs of the base engage a different one of the plurality of recesses of the monitor instrument when the monitor instrument is secured to the base.

10. The system of claim 7, wherein:
the base further comprises a bottom surface configured to face toward the subject's wrist and a top surface configured to face away from the subject's wrist;
the monitor instrument further comprises a top surface and a bottom surface, the bottom surface of the monitor instrument configured to face toward the top surface of the base of the wearable assembly when the monitor instrument and base are secured to one another;
the plurality of electrical contacts of the base are arranged on or proximate to the top surface of the base; and
the plurality of electrical contacts of the monitor instrument are arranged on or proximate to the bottom surface of the monitor instrument.

11. The system of claim 10, wherein the monitor instrument further comprises a display positioned on the top surface of the monitor instrument and configured to display information relating to said at least one physiological parameter.

12. The system of claim 7, wherein the base further comprises a cable comprising a first end electrically connected to the pulse oximetry sensor and a second end electrically connected to the printed circuit board of the base, the cable configured to transmit said one or more signals from said pulse oximetry sensor to said printed circuit board.

13. The system of claim 12, wherein the cable is permanently connected to the base of the wearable assembly.

14. The system of claim 7, wherein the base further comprises a memory in communication with said printed circuit board and configured to store information relating to said pulse oximetry sensor.

15. A system for monitoring physiological information of a subject, the system comprising:
a wearable assembly configured to be secured to the subject, the wearable assembly comprising:
a strap and a base, the strap configured to secure the base to a portion of a body of the subject, the base comprising a printed circuit board and a plurality of electrical contacts in electrical communication with the printed circuit board; and
a pulse oximetry sensor in electrical communication with said printed circuit board and configured to generate one or more signals indicative of at least one physiological parameter of the subject; and
a monitor instrument configured to removably secure to the base of the wearable assembly, the monitor instrument comprising:
a plurality of electrical contacts configured to contact the plurality of electrical contacts of the base of the wearable assembly and receive said one or more signals from the plurality of electrical contacts of the base when the monitor instrument and base are secured to one another; and
a processor configured to receive said one or more signals from the plurality of electrical contacts of the monitor instrument and determine said at least one physiological parameter of the subject;
wherein the monitor instrument is configured to wirelessly transmit said determined at least one physiological parameter of the subject to an external device.

16. The system of claim 15, wherein:
the base further comprises:
a bottom surface configured to face toward the portion of the subject's body and a top surface configured to face away from the portion of the subject's body; and
a plurality of tabs extending generally perpendicular relative to said top surface of the base;
the monitor instrument further comprises:
a top surface and a bottom surface, the bottom surface of the monitor instrument configured to face toward the top surface of the base of the wearable assembly when the monitor instrument and base are secured to one another; and
a plurality of recesses; and
each of the plurality of tabs of the base engage a different one of the plurality of recesses of the monitor instrument when the monitor instrument is secured to the base.

17. The system of claim 15, wherein:
the base further comprises a bottom surface configured to face toward the portion of the subject's body and a top surface configured to face away from the portion of the subject's body;
the monitor instrument further comprises a top surface and a bottom surface, the bottom surface of the monitor instrument configured to face toward the top surface of the base of the wearable assembly when the monitor instrument and base are secured to one another;
the plurality of electrical contacts of the base are arranged on or proximate to the top surface of the base; and
the plurality of electrical contacts of the monitor instrument are arranged on or proximate to the bottom surface of the monitor instrument.

18. The system of claim 17, wherein the monitor instrument further comprises a display positioned on the top surface of the monitor instrument and configured to display information relating to said at least one physiological parameter.

19. The system of claim 15, wherein the base further comprises a cable comprising a first end electrically connected to the pulse oximetry sensor and a second end electrically connected to the printed circuit board of the base, the cable configured to transmit said one or more signals from said pulse oximetry sensor to said printed circuit board.

20. The system of claim 15, wherein the base further comprises a memory in communication with said printed circuit board and configured to store information relating to said pulse oximetry sensor.
